SEQUENCE LISTING (5 sequences)

SEQ ID NO: 1 - DNA, 2713 bp, Homo sapiens
cggtggctgggctgcgcttgggtccgtcgctgcttcggtgtccctgtcgggcttcccagcagcggcctagcgggaaaagtaaaagatgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataagtcgggcagtggttttaatggaggctttggctcaagcatggattctaagtcttctggctggggaatgtagacagtggggttgtggttggttggtatagaatggtgggaattcaaatttttctaaactcatggtaagtatattgtaaaatacatatgtactaagaattttcaaaattggtttgttcagtgtggagtatattcagcagtatttttgacatttttctttagaaaaaggaagagctaaaggaattttataagttttgttacatgaaaggttgaaatattgagtggttgaaagtgaactgctgtttgcctgattggtaaaccaacacactacaattgatatcaaaaggtttctcctgtaatattttatccctggacttgtcaagtgaattctttgcatgttcaaaacggaaaccattgattagaactacattctttaccccttgttttaatttgaaccccaccatatggatttttttccttaagaaaatctccttttaggagatcatggtgtcacagtgtttggttcttttgttttgttttttaacacttgtctcccctcatacacaaaagtacaatatgaagccttcatttaatctctgcagttcatctcatttcaaatgtttatggaagaagcacttcattgaaagtagtgctgtaaatattctgccataggaatactgtctacatgctttctcattcaagaattcgtcatcacgcatcacaggccgcgtctttgacggtgggtgtcccatttttatccgctactctttatttcatggagtcgtatcaacgctatgaacgcaaggctgtgatatggaaccagaaggctgtctgaacttttgaaaccttgtgtgggattgatggtggtgccgaggcatgaaaggctagtatgagcgagaaaaggagagagcgcgtgcagagacttggtggtgcataatggatattttttaacttggcgagatgtgtctctcaatcctgtggctttggtgagagagtgtgcagagagcaatgatagcaaataatgtacgaatgttttttgcattcaaaggacatccacatctgttggaagacttttaagtgagtttttgttcttagataacccacattagatgaatgtgttaagtgaaatgatacttgtactccccctacccctttgtcaactgctgtgaatgctgtatggtgtgtgttctcttctgttactgatatgtaagtgtggcaatgtgaactgaagctgatgggctgagaacatggactgagcttgtggtgtgctttgcaggaggacttgaagcagagttcaccagtgagctcaggtgtagttctgtgtcctgtgcttggatgctttttataagagttgtcattgttggaaattcttaaataaaactgatttaaataaaaaaaaaaaaaaagggcggccgcccttttttttttttttttagagcttttatatttgcgtttattcttcatttaactttttaaaacactactatagtttattaaaaaaaaaaaa

SEQ ID NO: 2 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 3 - protein, 8 aa, Homo sapiens
VFIPKPFR

SEQ ID NO: 4 - protein, 414 aa, Homo sapiens; identical to SEQ ID NO: 2

SEQ ID NO: 5 - RNA, 19 nt, Homo sapiens
ggccacuccugcaucuuua

(19) United States
(12) Patent Application Publication
(10) Pub. No.: US 2010/0136573 A1
(43) Pub. Date: Jun. 3, 2010

(54) DIAGNOSING NEURODEGENERATIVE DISEASES

(76) Inventors: Leonard Petrucelli, Ponte Vedra Beach, FL (US); Yongjie Zhang, Jacksonville, FL (US); Yafei Xu, Jacksonville, FL (US); Tania Gendron, Jacksonville, FL (US)

Correspondence Address: FISH & RICHARDSON P.C., PO BOX 1022, MINNEAPOLIS, MN, 55440-1022, US

(21) Appl. No.: 12/627,851

(22) Filed: Nov. 30, 2009

Related U.S. Application Data
(63) Continuation-in-part of application No. PCT/US2008/065377, filed on May 30, 2008.
(60) Provisional application No. 60/932,656, filed on Jun. 1, 2007.

(51) Int. Cl. G01N 33/53; C07K 16/00

(52) U.S. Cl. 435/7.1; 530/387.9

(57) ABSTRACT

This document provides methods and materials related to determining whether or not a mammal (e.g., a human) has a neurodegenerative disease (e.g., frontotemporal dementia, AD, or amyotrophic lateral sclerosis (ALS)). For example, methods and materials for using the levels of TDP-43 polypeptides and/or TDP-43 polypeptide cleavage products (e.g., 25 kD and 35 kD TDP-43 polypeptide cleavage products) in a biological fluid (e.g., cerebrospinal fluid) to determine whether or not a mammal has a neurodegenerative disease (e.g., frontotemporal dementia, AD, or ALS) are provided.

### CROSS-REFERENCE TO RELATED APPLICATIONS

This application is a continuation-in-part application of International PCT Application Serial No. PCT/US2008/065377, filed May 30, 2008, which claims the benefit of U.S. Provisional Application Ser. No. 60/932,656, filed Jun. 1, 2007, each of which are hereby incorporated by reference in their entirety.

### BACKGROUND

1. Technical Field

This document relates to methods and materials involved in determining whether or not a mammal (e.g., human) has a neurodegenerative disease (e.g., Alzheimer's disease).

2. Background Information

Many people are diagnosed with a neurodegenerative disease such as Alzheimer's disease (AD). In fact, AD is the most common form of age-related neurodegenerative illness. The defining pathological hallmarks of AD are the presence of neurofibrillary tangles and senile plaques in the brain. Amyloid β polypeptides (Aβ) are the major constituents of amyloid plaques and are derived from altered processing of amyloid precursor proteins (APPs).

### SUMMARY

This document provides methods and materials related to determining whether or not a mammal (e.g., a human) has a neurodegenerative disease (e.g., frontotemporal dementia, AD, or amyotrophic lateral sclerosis (ALS)). TAR DNA binding protein (TDP-43) is a polypeptide that can be cleaved into fragments of 25 kD and 35 kD. As described herein, the levels of TDP-43 polypeptides and TDP-43 polypeptide cleavage products (e.g., 25 kD and 35 kD TDP-43 polypeptide cleavage products) in a biological fluid (e.g., cerebrospinal fluid, serum, or plasma) can be measured to determine whether or not a mammal has a neurodegenerative disease (e.g., frontotemporal dementia, AD, or ALS). Mammals having an elevated level of a TDP-43 polypeptide or a TDP-43 polypeptide cleavage product (e.g., a 25 kD or 35 kD TDP-43 polypeptide cleavage product) within a biological fluid can be classified as having a neurodegenerative disease. Determining whether or not a mammal has a neurodegenerative disease can help mammals receive proper treatment or medical care. For example, determining whether or not a human has a neurodegenerative disease can help clinicians determine proper treatment and medical care options for the human.

In general, one aspect of this document features a method for assessing a mammal for a neurodegenerative disease. The method comprises, or consists essentially of, determining whether or not a biological fluid from the mammal contains an elevated level of a TDP-43 polypeptide or a TDP-43 polypeptide cleavage product, wherein the presence of the elevated level indicates that the mammal has the neurodegenerative disease. The mammal can be a human. The neurodegenerative disease can be frontotemporal dementia, Alzheimer's disease, or amyotrophic lateral sclerosis. The biological fluid can be a cerebrospinal fluid, serum, or plasma. The method can comprise determining whether or not the biological fluid from the mammal contains an elevated level of the TDP-43 polypeptide. The elevated level of the TDP-43 polypeptide can be greater than 10 ng/mL. The method can comprise determining whether or not the biological fluid from the mammal contains an elevated level of the TDP-43 polypeptide cleavage product. The elevated level of the TDP-43 polypeptide cleavage product can be greater than 10 ng/mL. The method can comprise obtaining the biological fluid from the mammal. The mammal can comprise the elevated level, and wherein the method can comprise classifying the mammal as having the neurodegenerative disease. An anti-TDP-43 polypeptide antibody can be used to determine whether or not the biological fluid from the mammal contains the elevated level. The TDP-43 polypeptide cleavage product can be about 25 kD. The TDP-43 polypeptide cleavage product can be about 35 kD. An antibody can be used to determine whether or not said biological fluid from the mammal contains the elevated level. The antibody can recognize a human TDP-43 polypeptide cleavage product that is about 25 kD. The antibody can lack the ability to recognize a full length human TDP-43 polypeptide. The antibody can be produced using the sequence set forth in SEQ ID NO:3.

In another aspect, this document features an antibody comprising the ability to recognize a human TDP-43 polypeptide cleavage product that is about 25 kD, wherein the antibody does not recognize a full length human TDP-43 polypeptide. The antibody can be produced using the sequence set forth in SEQ ID NO:3.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1. Proteolytic processing of TDP-43. (a) Progranulin knockdown and overexpression (O.E.) in HeLa cells using siRNA. (b) PGRN immunocytochemistry demonstrated mostly cytoplasmic and some nuclear staining in HeLa cells. (c) Schematic representation of the amino acid sequence of TDP-43 (GenBank Accession No. NP_{—}031401; SEQ ID NO:4), depicting the three casapse-3 recognition motifs, WOW (bold and underlined), generating fragments with an approximate molecular weight (kD) of 42 (DEND . . . ), 35 (DETD . . . ), and 25 (DVMD . . . ) molecular weight (kD). (d) In vitro cleavage of recombinant TDP-43 polypeptide. GST-tagged TDP-43 polypeptide (2 μg) was incubated with purified cleaved caspase-3, (Chemicon, 2 units) for 2 and 4 hours. Samples were separated on 10% SDS/PAGE and stained with Coomassie. (e) In vitro generation of pathologic TDP-43 by cells treated with PGRN RNAi, but not by cells treated with control RNAi. Progranulin deficiency leads to an increase in cleaved caspase-3 activity. Treatment with a pan-caspase inhibitor suppresses progranulin-mediated TDP-43 cleavage and caspase-3 activity. (f) Biochemical analyses of TDP-43 in sporadic and familial FTLD-U harboring Gly333ValfsX28 (lane #3) and Asp22ArgfsX43 (lane #4) (Gass et al., Hum. Mol. Genet., 15:2988-3001 (2006)) and AD brains. Immunoblots of urea fractions from temporal cortex of FTLD-U patients with rabbit anti-TDP-43 antibody revealed a similar pathological profile of TDP-43 similar to progranulin knockdown experiments (FIG. 1c), but not in Alzheimer's disease brain or controls. Pathologic ˜25-kD (*) bands, 35 (**) kD bands, and 45-kD bands (***) are indicated.

FIG. 2. Cleavage, solubility, and cellular localization of TDP-43. (a) HeLa cells treated with staurosporine (0.2 μM, 3 hours) revealed increased proteolytic cleavage of TDP-43 and cleaved casapse-3 activity. (b) Biochemical analyses of TDP-43 in HeLa cells. Immunoblots of cell extracts treated with PRGN RNAi and staurosporine with rabbit anti-TDP-43 antibody revealed the pathologic ˜25-kD and ˜35 bands in the triton-insoluble fraction. (c) Cytoplasmic translocation of TDP-43. Immunofluorescent staining for endogenous TDP-43 or histone in HeLa cells treated with either vehicle or staurosporine followed by immunofluorescent staining with anti-TDP-43 antibody (green; color not shown) or anti-histone (green; color not shown) and examined by confocal microscopy. The nucleus was stained with DAPI (blue; color not shown). Scale bar, 20 μm. TDP-43 immunohistochemistry of motor neurons in ALS showing normal nuclear but no cytoplasmic labeling (e); early pathologic cytoplasmic, but no nuclear labeling (f); and fully formed fibrilliar cytoplasmic inclusions with no nuclear labeling (g). This illustrates pathologic redistribution of TDP-43 in vulnerable neurons. Magnification, 40×.

FIG. 3. Proteolytic processing and distribution of TDP-43. (a) Progranulin knockdown and staurosporine treatment in H4 neuroglioma cells demonstrating increase pathologic cleavage. (b) TDP-43 immunocytochemistry demonstrated mostly cytoplasmic and some nuclear staining in H4 cells treated with staurosporine.

FIG. 4 is a photograph of a Western blot of control and AD CSF (25 μg total protein) immunoblotted with rabbit-anti-TDP-43 antibody. Western blot revealed an increase in total TDP-43 levels (full-length, *) and a truncated TDP-43 (35 kD, **) species in a subset of AD patients. Hela cell line extract treated with staurosporine served as a positive control (“+”).

FIG. 5 is a sequence listing of a nucleotide sequence (SEQ ID NO:1) that encodes a human TDP-43 polypeptide.

FIG. 6 is a sequence listing of an amino acid sequence of a human TDP-43 polypeptide (SEQ ID NO:2).

FIG. 7 is a photograph of a Western blot demonstrating that proteasome inhibition increases the proteolytic cleavage of TDP-43. Western blot analyses of H4 neuroglioma cells treated with the proteasome inhibitor, PSI (10 μM, 24 hours) and a pan-caspase inhibitor, Z-VAD-FMK (100 μM, 24 hours) separately or in combination. Treatment with PSI revealed a reduction in full-length TDP-43 (light exposure), an increase in proteolytic cleavage of TDP-43 fragments (35 kD and 25 kD), and an increase in caspase-3 activity. Treatment with a pan-caspase inhibitor suppressed PSI-induced TDP-43 cleavage and caspase-3 activity. HSP70 levels were increased after PSI treatment, and the levels persisted in the presence of a pan-caspase inhibitor. Similar results were obtained in three independent experiments.

FIG. 8 is a photograph of tissue from an FTLDu patient immunostained with a polyclonal antibody designated MC2085 and raised against the 25 kD fragment of TDP-43. This antibody detected cytoplasmic inclusions in the hippocampus (left) and neuritic staining in the cortex (right). No nuclear staining was observed with this antibody.

FIG. 9. C-terminal caspase-dependent fragments form cytoplasmic, ubiquitin-positive inclusions. (A) Schematic representation of the TDP-43 molecule and various TDP-43 constructs. The “X” symbol indicates two of the caspase-cleavage sites which result in either 35 or 25 kDa C-terminal TDP-43 fragments. GFP, green fluorescent protein; NLS, nuclear localization signal; RRM, RNA recognition motif; GRR, glycine rich region. (B) Expression of GFP fusion proteins in vitro. HEK293 were transfected with one of the constructs for 48 hours, and cell lysates were subjected to Western blot analysis using an anti-GFP antibody to compare expression levels of the fusion proteins. The membrane was stripped and re-probed with an anti-GAPDH antibody to verify protein loading. (C-K) Fluorescent confocal microscopy demonstrates the diffuse cytoplasmic and nuclear distribution of GFP (C) and GFP-TDP-ΔNR1 (F), and the predominantly nuclear localization of wild-type GFP-TDP-43 (D) and the caspase-resistant mutant (E). The cytoplasmic inclusions formed in cells transfected with GFP-TDP-35 (G) or GFP-TDP-25 (H). Inclusions formed from the 25 kDa fragment persist after treatment with 0.2% Triton X-100 (I) or nocodazole (J). (K) Ubiquitin immunostaining reveals that cytoplasmic inclusions formed from GFP-TDP-25 are ubiquitin-positive. Scale bars, 10 μm.

FIG. 10. The 25 kDa C-terminal fragment of TDP-43 is prone to phosphorylation but its phosphorylation is not required for inclusion formation. (A) Cells were transfected for GFP-TDP-43 or GFP-TDP-25 then immunostained with anti-pTDP-43, which detects TDP-43 when phosphorylated at S409/S410. Fluorescent confocal microscopy demonstrates enhanced staining of GFP-TDP-25 compared to GFP-TDP-43. (B) Lysates from HEK293 cells transfected with GFP-TDP-43, GFP-TDP-43NLSmut, GFP-TDP-25, or GFP-TDP-255409A/S410A were subjected to Western blot analysis and probed with anti-pTDP-43. The 25 kDa C-terminal fragment exhibited marked anti-pTDP-43 immunoreactivity compared to GFP-TDP-43 or GFP-TDP-43NLSmut. GFP-TDP-25S409A/S410A did not exhibit any immunoreactivity. The membrane was re-probed with anti-GAPDH to verify protein loading. (C) Fluorescent confocal microscopy demonstrates that GFP-TDP-25S409A/S410A, which is not phosphorylated at S409/S410, can form cytoplasmic inclusions.

FIG. 11. The 25 kDa C-terminal fragment enhances cellular toxicity. (A) The release of LDH into the media was used as an indicator of cell toxicity. LDH levels were measured 72 hours after cells were transfected with the indicated constructs. Data from three separate experiments was analyzed by one-way analysis of variance followed by Tukey's post-hoc analysis (** p<0.001). (B) Increased apoptosis in differentiated M17 neuroblastoma cells expressing GFP-TDP-25 compared to cells expressing GFP alone or GFP-TDP-43. Cultures were fixed and stained with Hoechst to label nuclei (which can be observed as a blue stain) and activated caspase-3 antibody (which can be observed as a red stain). Scale bar=10 μM.

FIG. 12. The 25 kDa C-terminal fragment does not affect full-length TDP-43 function or cellular localization. (A, B) To examine the effect of C-terminal fragments on endogenous TDP-43 function, cells were co-transfected with a CFTR minigene construct and a vector encoding the GFP-fusion proteins, as indicated (A). To examine the effect of C-terminal fragments on exogenous TDP-43, cells were also transfected with a vector for Flag-TDP-43 (B). Two days after transfection, the exon inclusion and exclusion products were examined by RT-PCR. The schematic shown to the left of gels in A and B depict transcripts containing or lacking exon 9 with or without a cryptic splice variant (grey box). Black boxes depict non-CFTR exons. Top and lower bands correspond to transcripts that include or exclude exon 9, respectively. The middle band represents the cryptic splice variant. TDP-25 inhibited neither endogenous nor exogenous TDP-43 exon exclusion activity. GFP-TDP76-414 did markedly attenuate exogenous TDP-43 activity, despite its nuclear localization (C), which was examined by staining cells with the nuclear marker, Hoechst (blue), after transfection. Scale bar=10 μM. (D) Fluorescent confocal microscopy reveals that GFP-TDP-25 inclusions fail to sequester wild-type Flag-TDP-43. HEK293 cells were co-transfected with Flag-TDP-43 and GFP-TDP-43 or GFP-TDP-25 and stained with Hoescht (which can be observed as a blue stain) and anti-Flag antibody (which can be observed as a red stain). Scale bar=10 μM. (E-F) Co-immunoprecipitation experiments reveal that GFP-TDP-25 and GFP-TDP76-414 bind strongly to hnRNPA2 (E), but only weakly to wild-type Flag-TDP-43 (F). GFP-TDP-43 and the C-terminal deletion products, GFP-TDP1-257 and GFP-TDP1-175, bind Flag-TDP-43, indicating that TDP-43 molecules interact and that their binding to one another is enhanced by the presence of the N-terminal. (* represents a non-specific band).

FIG. 13. A conformation-dependent TDP-43 antibody that detects C-terminal fragments is specific for pathologic inclusions in human TDP-43 proteinopathies. (A) Three-dimensional modeling of wild-type TDP-43 (amino acids 101-264). The positions of the eight amino acid residues following the caspase cleavage motif, selected to generate a polyclonal antibody, are indicated. Aspartate 219, which is part of the caspase cleavage motif, is buried within the molecule (arrow). (B) Western blot analysis of lysates from HEK293 cells transfected with GFP only, GFP-TDP-43, or GFP-TDP-25 and probed with MC2085. *Indicates endogenous TDP-43. (C) MC2085 immunostaining of HEK293 cells transfected with GFP-TDP-43 or GFP-TDP-25. Note the absence of staining in cells expressing wild-type TDP-43 compared to those expressing the 25 kDa C-terminal fragment. Scale bars, 10 gm. (D-E) Immunostaining of tissue from FTLD-U brain (D) and ALS brain (E) with MC2085 shows cytoplasmic inclusions with little or no nuclear TDP-43. Magnification 40×.

FIG. 14. The N-terminal TDP-43 antibody, MC2079, does not detect GFP-TDP-25 inclusions. (A) Western blot analysis of lysates from HEK293 cells transfected with either a vector for GFP, GFP-TDP-43, or GFP-TDP-25 and probed with the N-terminal antibody, MC2079. (B) MC2079 immunostaining of HEK293 cells transfected with GFP-TDP-25. The inclusions are not positive for MC2079.

FIG. 15. A range of anti-pTDP-43 and anti-cTDP immunoreactive inclusions are present in hypoglossal motor neurons in ALS.

FIG. 16. Non-purified anti-pTDP-43 specifically detects TDP-43 when phosphorylated at S409/S410.

FIG. 17. Affinity purified anti-pTDP-43 detects phosphorylated GFP-TDP₂₂₀₋₄₁₄ and GFP-TDP-43 from cell lysates (A), as well as phosphorylated TDP-43 in the urea fraction from FTLD-U, but not control, human brain tissue (B).

FIG. 18. Anti-pTDP-43 detects recombinant GST-TDP-43 following in vitro phosphorylation by casein kinase 1 but does not detect non-phosphorylated GST-TDP-43. ** Indicates the shift in molecular weight due to phosphorylation of GST-TDP-43. The arrows indicate the position of non-phosphorylated GST-TDP-43 which is detected by anti-TDP-43 (ProteinTech) but not by anti-pTDP-43.

FIG. 19. Detection of phospho-GST-TDP-43 by sandwich ELISA using anti-pTDP-43 and a commercially available mouse monoclonal TDP-43 antibody (2E2-D3, Novus).

FIG. 20. Standard curve using recombinant GST-TDP-43. A sandwich ELISA was performed using the mouse monoclonal TDP-43 antibody, 2E2-D3 (Novus), as the capture antibody, and the rabbit polyclonal antibody, 10782-2-AP (ProteinTech) as the detection antibody. GST-TDP-43 was used as the antigen. Note that the absorbance values for the lower concentrations of GST-TDP-43 are magnified in panel B.

### DETAILED DESCRIPTION

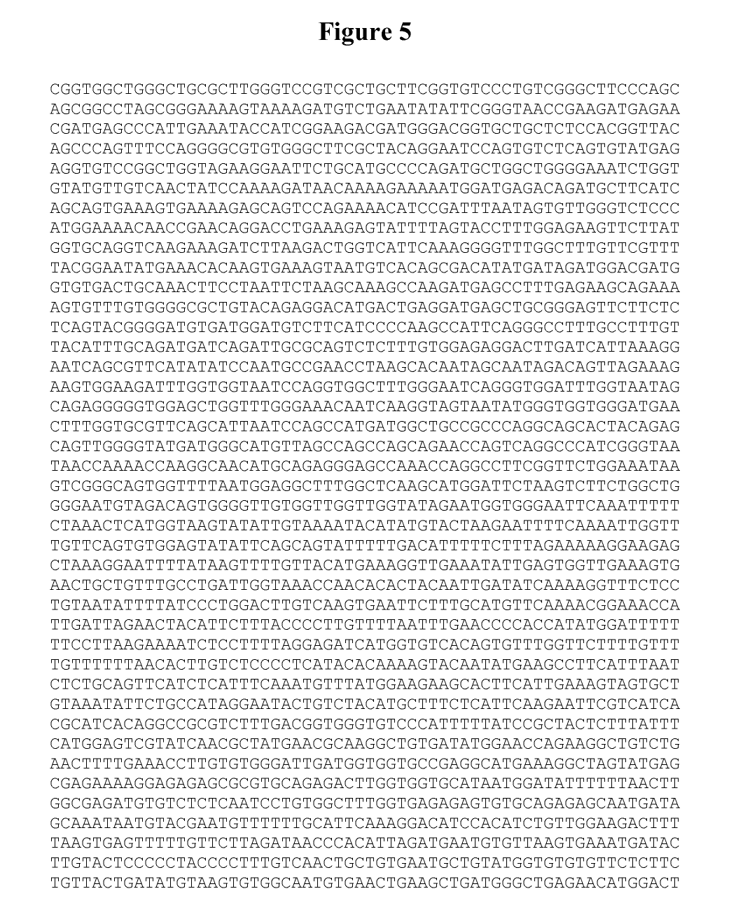

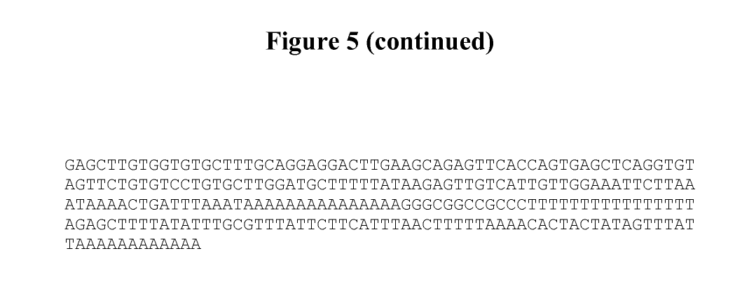

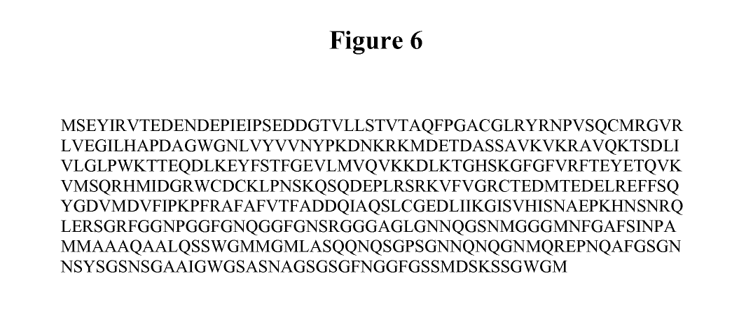

This document provides methods and materials related to determining whether or not a mammal has a neurodegenerative disease (e.g., frontotemporal dementia, AD, amyotrophic lateral sclerosis (ALS), or Parkinson's disease). For example, this document provides methods and materials for determining whether or not a biological fluid from a mammal contains an elevated level of a TDP-43 polypeptide or a TDP-43 polypeptide cleavage product. As described herein, if the level of a TDP-43 polypeptide or a TDP-43 polypeptide cleavage product in a biological fluid (e.g., cerebrospinal fluid, serum, or plasma) from a mammal is elevated, then the mammal can be classified as having a neurodegenerative disease. In some cases, a detectable level of a TDP-43 polypeptide or a TDP-43 polypeptide cleavage product in a biological fluid (e.g., cerebrospinal fluid) from a mammal can indicate that that mammal has a neurodegenerative disease. A human TDP-43 polypeptide can have the amino acid sequence set forth in FIG. 6 and can be encoded by a nucleic acid molecule having the nucleotide sequence set forth in FIG. 5.

In some cases, a mammal suspected to have a neurodegenerative disease can be evaluated by assessing the levels of TDP-43 polypeptides and TDP-43 polypeptide cleavage products in a biological fluid to determine whether or not the mammal has a neurodegenerative disease. Any appropriate method can be used to identify a mammal as being suspected of having a neurodegenerative disease.

Any mammal can be assessed for a neurodegenerative disease using the methods and materials provided herein. For example, a human, cat, dog, or horse can be evaluated by assessing the levels of TDP-43 polypeptides and TDP-43 polypeptide cleavage products in a biological fluid to determine whether or not the mammal has a neurodegenerative disease. In some cases, a human suspected to have Alzheimer's Disease (AD) or FTLD-U can be assessed. In some cases, a human between the ages of about 30-65 years old can be assessed. In some cases, a human older than about 60 years of age can be assessed. In some cases, a human less than about 40 years of age can be assessed.

The term “elevated level” as used herein with respect to the level of a TDP-43 polypeptide or a TDP-43 polypeptide cleavage product is any level that is above a median level of a TDP-43 polypeptide or a TDP-43 polypeptide cleavage product, respectively, in a biological fluid (e.g., cerebrospinal fluid) from a random population of mammals (e.g., a random population of 10, 20, 30, 40, 50, 100, or 500 mammals) that do not have a neurodegenerative disease. In some cases, an elevated level of a TDP-43 polypeptide or a TDP-43 polypeptide cleavage product can be any detectable level of a TDP-43 polypeptide or a TDP-43 polypeptide cleavage product, respectively, in biological sample.

In some cases, an elevated level of a TDP-43 polypeptide or a TDP-43 polypeptide cleavage product can be a level of a TDP-43 polypeptide or a TDP-43 polypeptide cleavage product in a biological fluid from a mammal that is above a median level of the TDP-43 polypeptide or TDP-43 polypeptide cleavage product in a biological fluid from a random population of mammals (e.g., a random population of 10, 20, 30, 40, 50, 100, or 500 mammals) lacking a neurodegenerative disease that are of the same species, that are in the same age range (e.g., 30-65 years old, older than 60 years of age, less than 40 years of age, 25-40 years old, 40-50 years old, 50-60 years old, 60-70 years old, or 70-80 years old), that are of the same sex, and, in the case of humans, that are of the same race as the mammal being evaluated.

It will be appreciated that TDP-43 polypeptide and TDP-43 polypeptide cleavage product levels from comparable samples (e.g., cerebrospinal fluid) are used when determining whether or not a particular level is elevated. For example, a level of TDP-43 polypeptide cleavage product in cerebrospinal fluid from a particular species of mammal is compared to the median level of TDP-43 polypeptide cleavage product in cerebrospinal fluid from a population of mammals of the same species that do not have a neurodegenerative disease. In addition, TDP-43 polypeptide cleavage product levels can be compared to a median TDP-43 polypeptide cleavage product level measured using the same or a comparable method. In some cases, an elevated level of a TDP-43 polypeptide can be at least 0.1 ng/mL (e.g., at least 0.5 ng/mL, at least 1 ng/mL, at least 5 ng/mL, at least 10 ng/mL, at least 20 ng/mL, at least 50 ng/mL, at least 100 ng/mL, at least 150 ng/mL, at least 200 ng/mL, at least 500 ng/mL, at least 1 μg/mL, at least 2.5 μg/mL, at least 5 μg/mL, at least 10 μg/mL, at least 100 μg/mL, or more). In some cases, an elevated level of a TDP-43 polypeptide cleavage product can be at least 0.1 ng/mL (e.g., at least 0.5 ng/mL, at least 1 ng/mL, at least 5 ng/mL, at least 10 ng/mL, at least 20 ng/mL, at least 50 ng/mL, at least 100 ng/mL, at least 150 ng/mL, at least 200 ng/mL, at least 500 ng/mL, at least 1 μg/mL, at least 2.5 μg/mL, at least 5 μg/mL, at least 10 μg/mL, at least 100 μg/mL, or more).

Examples of biological fluids include, without limitation, cerebrospinal fluid, serum, and plasma. A biological fluid can be obtained from a mammal by any appropriate method. For example, cerebrospinal fluid can be obtained via spinal tap.

Any appropriate method can be used to determine the level of TDP-43 polypeptides and TDP-43 polypeptide cleavage products in a biological fluid from a mammal. For example, mass spectrometry can be used to determine the level of TDP-43 polypeptide cleavage products in a biological fluid. In some cases, the level of TDP-43 polypeptides and TDP-43 polypeptide cleavage products can be detected using a method that relies on an anti-TDP-43 polypeptide antibody. Such methods include, without limitation, FACS, Western blotting, ELISA, immunohistochemistry, and immunoprecipitation. An anti-TDP-43 polypeptide antibody can be labeled for detection. For example, an anti-TDP-43 polypeptide antibody can be labeled with a radioactive molecule, a fluorescent molecule, or a bioluminescent molecule. TDP-43 polypeptides and TDP-43 polypeptide cleavage products can also be detected indirectly using a labeled antibody that binds to an anti-TDP-43 polypeptide antibody that binds to a TDP-43 polypeptide or a TDP-43 polypeptide cleavage product. An anti-TDP-43 polypeptide antibody can bind to a TDP-43 polypeptide or a TDP-43 polypeptide cleavage product at an affinity of at least 10⁴ mol⁻¹ (e.g., at least 10₅, 10⁶, 10⁷, 10⁸, 10⁹, 10¹⁰, 10¹¹, or 10¹² mol⁻¹). Anti-TDP-43 polypeptide antibodies are commercially available, e.g., from ProteinTech Group, Inc, (Chicago, Ill.).

In some cases, an anti-TDP-43 polypeptide cleavage product antibody can be used to determine the level of a TDP-43 polypeptide or a TDP-43 polypeptide cleavage product in a biological fluid from a mammal. Such anti-TDP-43 polypeptide cleavage product antibodies can recognize full length TDP-43 polypeptides, particular TDP-43 polypeptide cleavage products, or both full length TDP-43 polypeptides and particular TDP-43 polypeptide cleavage products. For example, an anti-TDP-43 polypeptide fragment antibody having the ability to recognize the ˜25 kD fragment of human TDP-43 and not full length human TDP-43 can be obtained and used as described herein. Such antibodies can be obtained using common antibody production techniques and particular amino acid segments. For example, a portion of the ˜25 kD fragment of human TDP-43 that follows the caspase cleavage site (DXXD), VFIPKPFR (SEQ ID NO:3), can be used to obtain antibodies (e.g., polyclonal or monoclonal antibodies) having the ability to recognize the ˜25 kD fragment of human TDP-43 and not full length human TDP-43.

Once the level of a TDP-43 polypeptide or a TDP-43 polypeptide cleavage product in a biological fluid from a mammal is determined, then the level can be compared to a median level or a cutoff level and used to determine whether or not the mammal has a neurodegenerative disease. If it is determined that a biological fluid from a mammal contains an elevated level of a TDP-43 polypeptide and/or a TDP-43 polypeptide cleavage product, then the mammal can be classified as having a neurodegenerative disease. In some cases, the level of a TDP-43 polypeptide or a TDP-43 polypeptide cleavage product in a biological fluid can be used in combination with one or more other factors to determine whether or not a mammal has a neurodegenerative disease. For example, a TDP-43 polypeptide cleavage product level in a biological fluid can be used in combination with a cognitive or memory test.

This document also provides methods and materials to assist medical or research professionals in determining whether or not a mammal has a neurodegenerative disease. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of a TDP-43 polypeptide or a TDP-43 polypeptide cleavage product in a biological fluid, and (2) communicating information about the level to that professional.

Any appropriate method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

The invention will be further described in the following examples, which do not limit the scope of the invention described in the claims.

### EXAMPLES

### Example 1

### Progranulin Mediates Caspase-3-Dependent Cleavage of TDP-43

### Cell Culture and Treatments

HeLa and H4 cells were grown in Opti-Mem plus 10% FBS and 1% pen-strep and passaged every 3-5 days based on 90% confluence. For progranulin small interfering RNA (siRNA) transfections, siRNA was predesigned by QIAGEN (QIAGEN Inc, Valencia, Calif.) for GenBank Accession Number NM_{—}001012479, and the sense sequence was 5′-r(GGCCACUCCUGCAUCUUUA)dTdT-3′ (SEQ ID NO:5). siRNA experiments were carried out in 6-well plates. Final siRNA concentration (progranulin or a validated negative control siRNA) per well was 20 nM in Opti-Mem, with 4 μL of siLentFect transfection reagent (Bio-Rad, Hercules, Calif.) used per well. This mixture was incubated in a final volume of 500 μL for 20 minutes and then added to 40%-50% confluent HeLa and H4 cells in 6-well dishes plated the previous day for a final in-well volume of 2 mL. Seventy-two hours after transfection, cells were harvested for subsequent Western blot analysis in lysis buffer containing Co-IP buffer (50 mM Tris-HCl, pH 7.4, 1 M NaCl, 1% Triton-X-100, 5 mM EDTA) plus 1% SDS, PMSF, and both a protease and phosphatase inhibitor mixture. For caspase inhibitor treatment, the cells were transfected with progranulin siRNA for 24 hours, and then the pan-caspase inhibitor (Z-VAD-FMK) (EMD Chemicals, Inc. San Diego, Calif.) was added to cells for additional 48 hours at a final concentration of 100 μM. Cell lysates was prepared as described herein. For staurosorine treatment, 0.2 μM staurosorine (Cell Signaling, Beverly, Mass.) was added to cells 3 hours before the harvest.

### Fractionation Experiments

Briefly, cells were lysed in a buffer containing Co-IP buffer plus PMSF, and both a protease and phosphatase inhibitor mixture. After sonication, cells were centrifuged at 100,000 g at 4° C. for 30 minutes. Triton X-100 insoluble pellets were dissolved in the Co-IP buffer plus 1% SDS, PMSF, and both a protease and phosphatase inhibitor mixture. The soluble and insoluble fractions were used in western blot analysis.

The urea fraction of human tissue was prepared as described elsewhere (Neumann et al., J. Neuropathol. Exp. Neurol., 66:177-83 (2007)). Briefly, gray matter from FTLD-U postmortem cortex with progranulin mutation was dissected and weighed. Then, the tissue was extracted sequentially with low salt (LS) buffer, high salt-Triton (TX) buffer, myelin floatation buffer, and sarkosyl (SARK) buffer. The SARK insoluble materials were extracted in urea buffer and saved as urea. The urea fraction was used in western blot analysis.

### Western Blot Analysis

Protein concentrations of cells lysates were measured by a standard BCA assay (Pierce, Rockford, Ill.). Cell lysate samples were then heated in Laemmli's buffer, and equal amounts of protein were loaded into 10-well 10% or 4-20% Tris-glycine gels (Novex, San Diego, Calif.). After transfer, blots were blocked with 5% nonfat dry milk in TBST (TPS plus 0.1% Triton X-100) for 1 hour, and then the blots were incubated with rabbit polyclonal TDP-43 antibody (1:1000; ProteinTech Group, Inc, Chicago, Ill.), rabbit polyclonal progranulin antibody (1:1000; Zymed Laboratories, South San Francisco, Calif.), and rabbit polyclonal caspase-3 antibody (1:1000; Cell Signaling, Beverly, Mass.) or mouse monoclonal GAPDH antibody (1:5000; Biodesign International, Kennebunkport, Me.) overnight at 4° C. Membranes were washed three times for 10 minutes in TBST and then incubated with anti-mouse or anti-rabbit secondary antibodies conjugated to horseradish peroxidase (1:5000; Jackson ImmunoResearch, West Grove, Pa.) for 1 hour. Membranes were then washed three times for 10 minutes, and protein expression was visualized by ECL treatment and exposure to film.

### Immunofluorescence and Confocal Microscopy

HeLa or H4 cells were grown on glass coverslips for 24 hours and then treated with 1 μM staurosporine for 3 hours. After treatment with staurosporine, the cells were fixed with ice-cold methanol at −20° C. for 5 minutes and permeabilized with PBS-0.5% Triton X-100 for 10 minutes. After blocking with 5% BSA for 1 hour at 37° C., the cells were incubated overnight at 4° C. with rabbit polyclonal TDP-43 antibody (1:2000), rabbit polyclonal progranulin antibody (1:250), or rabbit polyclonal Histone H3 antibody (1:100; Cell Signaling, Beverly, Mass.), respectively. After washing, cells were incubated with the Oregon Green 488-conjugated goat anti-mouse IgG secondary antibody (TDP-43; 1:1000 or 1:500, progranulin and Histone H3) at 37° C. for 2 hours. Finally, Hoechst 33258 (1 μg/ml) was used to stain the nuclei. Images were obtained on a Zeiss (Thornwood, N.Y.) LSM 510 META confocal microscope.

### In Vitro Caspase-3 Assay

Recombinant human GST-TDP43 (2 μg) was incubated with active human recombinant caspase-3 (2 units, CHEMICON International, Inc., Temecula, Calif.) in reaction buffer containing 100 mM NaCl, 50 mM HEPES, 10 mM DTT, 1 mM EDTA, 10% glycerol, 0.1% CHAPS, pH 7.4) at 37° C. for 2 hours or 4 hours, respectively. Cleavage reactions were terminated by addition of 2×SDS loading buffer. Full-length or caspase-3-treated recombinant GST-TDP43 (0.5 μg) was separated by 10% SDS-PAGE and stained with Coomassie blue.

### Results

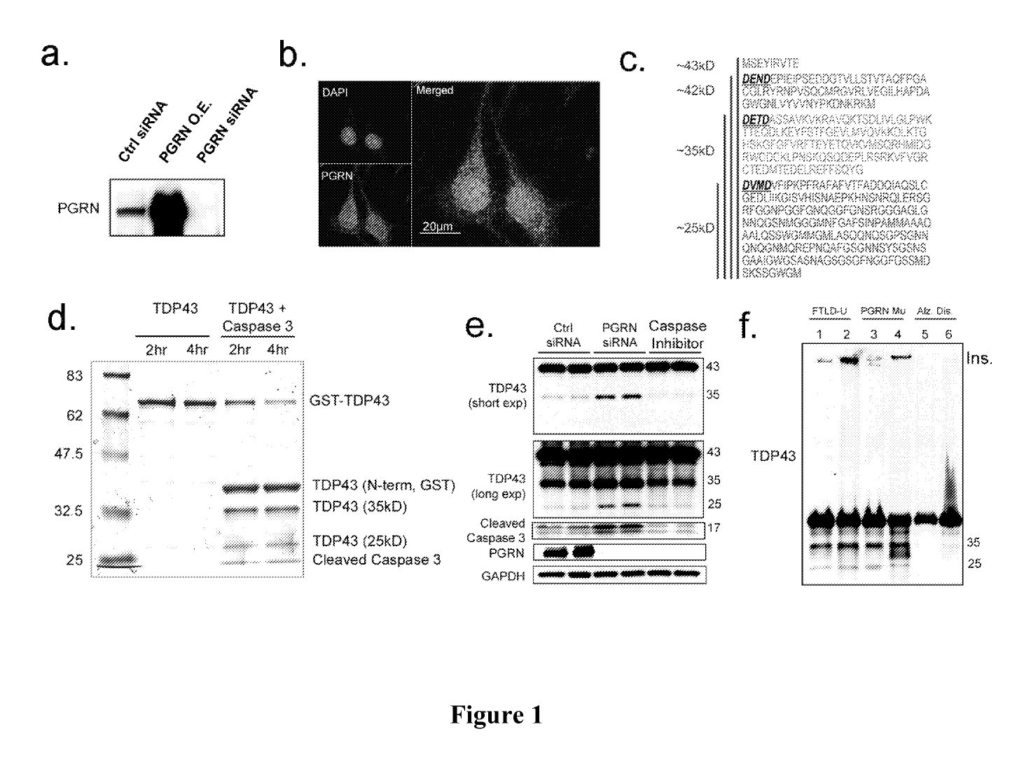

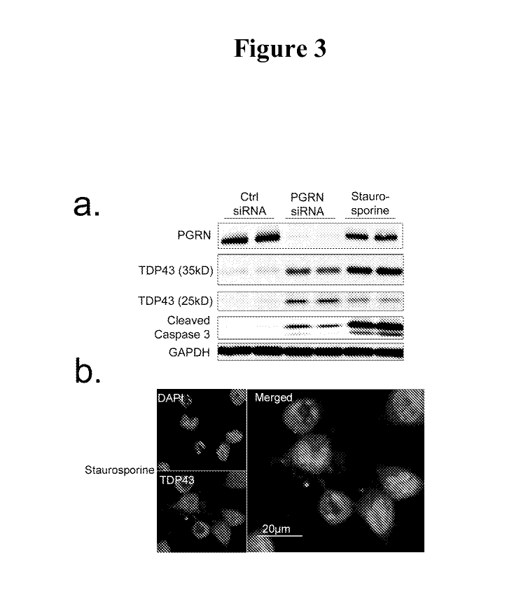

The involvement of progranulin in TDP-43 processing was evaluated. Two cell lines that have high endogenous levels of progranulin, HeLa and H4 neuroglioma, were treated with PGRN siRNA to reduce PGRN expression selectively. Treatment of both HeLa epithelial cells (FIG. 1a) and H4 neuroglioma cells (FIG. 3a) with PGRN RNAi markedly reduced PGRN expression and as a result no detectable progranulin band was observed on Western blots under these conditions compared to control siRNA. Endogenous progranulin was mostly present in the cytoplasmic compartment in these cells, consistent with in vivo data (FIG. 1b; Mackenzie et al., Brain, 129:3081-90 (2006) and Daniel et al., J. Histochem. Cytochem., 48:999-1009 (2000)).

Analysis of the amino acid sequence of TDP-43 revealed three putative caspase-3 cleavage consensus sites (DXXD; FIG. 1c). Caspase-3 cleavage at these sites would be predicted to produce approximately 42, 35, and 25 kD fragments. Since a DXXD consensus sequence was identified in the TDP-43 amino acid sequence, an in vitro assay was performed to determine if caspase-3 activity was able to cleave TDP-43. Recombinant GST-tagged TDP-43 (N-terminal tag, full length) was incubated with or without purified cleaved caspase-3 for 2 and 4 hours. Coomassie staining revealed the cleavage of full length TDP-43 by purified cleaved caspase-3, resulting in three distinct fragments of approximately 42, 35, and 25 kD (FIG. 1d).

To explore a potential mechanism of PGRN-knockdown-mediated cleavage of TDP-43, the levels of total caspase-3 under these conditions were examined. Cells treated with PGRN siRNA exhibited significantly increased levels of cleaved caspase-3 activity compared to control RNAi (FIGS. 1e and 3a), consistent with a report using neutralizing antibodies against progranulin (Liau et al., Cancer Res., 60:1353-60 (2000)).

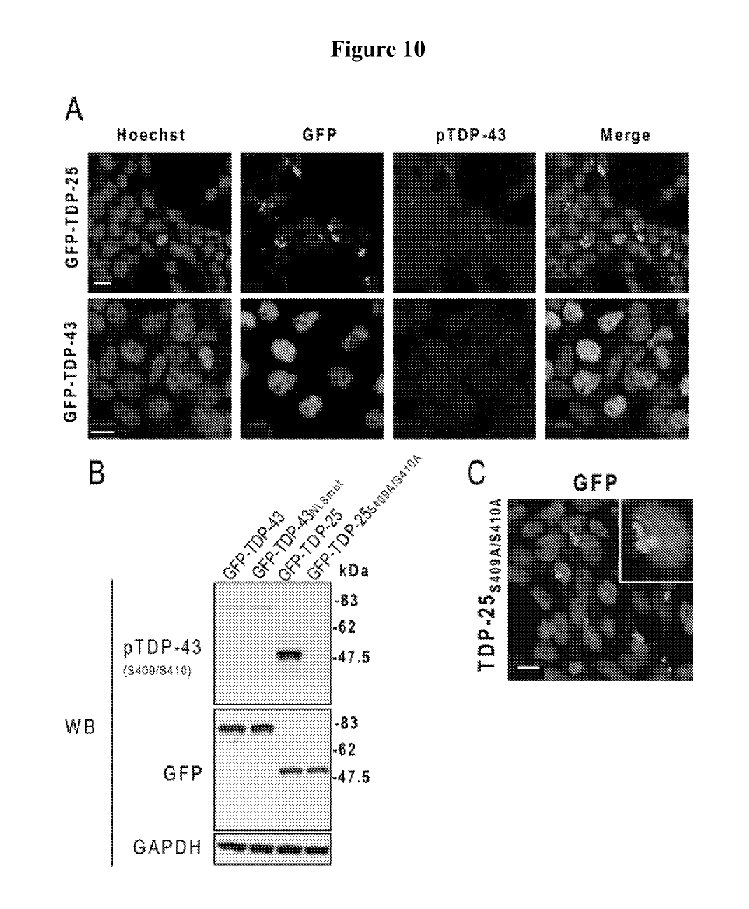

After demonstration of specific in vitro knockdown of progranulin and its affect on caspase-3 activity, whether progranulin deficiency was involved in the proteolytic processing of TDP-43 was investigated. HeLa epithelial cells (FIG. 1e) and H4 neuroglioma cell lines (FIG. 3a) were treated with either control siRNA or PGRN siRNA for 72 hours. This revealed that endogenous TDP-43 within these cells was cleaved into similar ˜35 and ˜25 kD fragments as those found in FTLD-U cases and the in vitro assay (FIGS. 1d and f). Their production was inhibited by the caspase inhibitor, Z-VAD (OMe)-FMK (FIG. 1e). Taken together, these results demonstrate that suppression of PGRN expression can be sufficient to promote proteolytic cleavage and accumulation of TDP-43 through a mechanism that implicated programmed cell death. The ˜35 and ˜25 kD fragments from these cell lysates indeed had a similar molecular mass and biochemical profile to TDP-43 extracted from brain of familial and sporadic FTLD-U with similar procedures (FIG. 10. In contrast, TDP-43 from Alzheimer's disease cases did not contain these fragments (FIG. 1f), nor did vascular dementia cases.

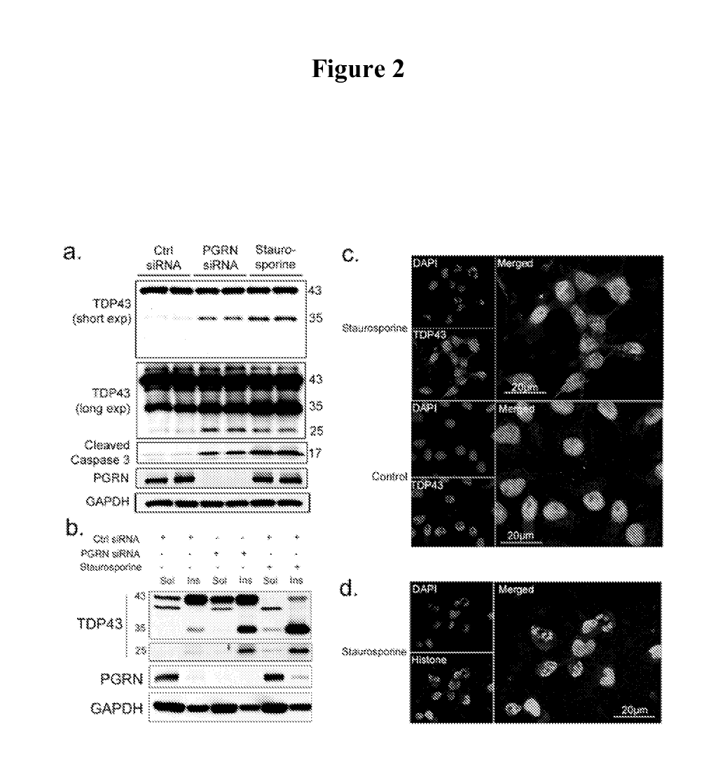

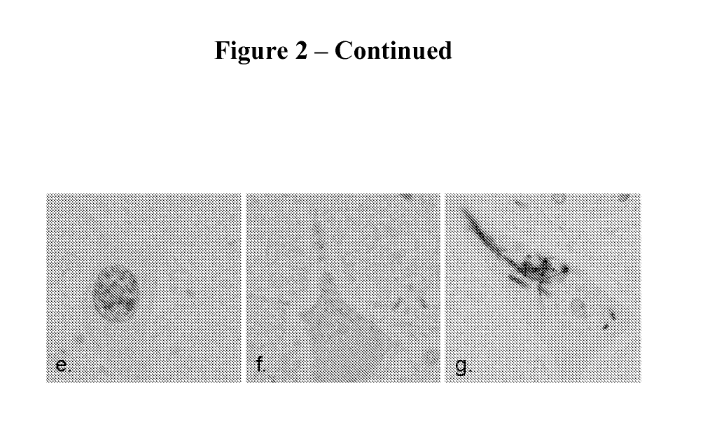

The evidence from these in vitro studies demonstrates that proteolytic cleavage of TDP-43 in FTLD-U may be mediated by casapse-3. To further confirm these results, HeLa and H4 cells were exposed to staurosporine, a potent inducer of apoptosis and caspase-3 activation. HeLa and H4 cells treated with staurosporine had increased cleavage of TDP-43 with a concomitant increase in caspase-3 activity (FIGS. 2a and 3a). In order to characterize the TDP-43 polypeptide biochemically, HeLa cell lysates from control siRNA-, PGRN siRNA-, and staurosporine-treated conditions were separated into triton soluble and insoluble fractions and analyzed by immunoblot. Whereas full-length TDP-43 polypeptide was present in both soluble and insoluble fractions under control conditions, the ˜25 and ˜35 kD bands were only detectable in triton insoluble fractions of PGRN siRNA- and staurosporine-treated cells (FIG. 2b). In contrast to untreated cells, which mostly exhibited strong nuclear localization (FIGS. 2c and 3b), staurosporine caused increased cytoplasmic TDP-43 staining Histones were localized to the nuclear compartment regardless of staurosporine treatment (FIG. 2d). These results indicate that a general disruption of the nuclear membrane was not caused by staurosporine, and thus did not likely contribute to passive leakage of TDP-43 into the cytoplasm. This pattern of TDP-43 redistribution can also be observed in FTLD-U and ALS, where vulnerable neurons (e.g., hypoglossal motor neurons in ALS) exhibit progressive redistribution of TDP-43 from the nucleus (FIG. 2e) to cytoplasm (FIG. 2f) and finally to fibrillar cytoplasmic inclusions (FIG. 2g).

The identification of TDP-43 as the major component of the neuropathological features observed in FTLD-U and ALS, and the determination that haploinsufficiency of progranulin leads to FTLD-U were pivotal findings for advancing the understanding of the dysfunctional pathways underlying these disorders (Neumann et al., J. Neuropathol. Exp. Neurol., 66:177-83 (2007)). The results provided herein demonstrate that haploinsufficiency of progranulin can lead to pathological processing of TDP-43 by caspase-3.

A high degree of similarity exists between the cell culture systems provided herein and human cases of FTLD-U. The activation of caspase-3 observed in the cell culture systems was consistent with reports demonstrating activated caspase-3 immunoreactivity in FTLD-U and ALS (Martin et al., J. Neuropathol. Exp. Neurol., 58:459-71 (1999) and Su et al., Exp. Neurol., 163:9-19 (2000)). The fragmentation of TDP-43 into 25 kD and 35 kD proteolytic species and changes in their solubility profile also was similar to biochemical properties of TDP-43 from FTLD-U brain tissue. The cell culture models provide herein can be used to screen for agents that can prevent pathological fragmentation of TDP-43 without affecting programmed cell death.

In summary, the results provided herein demonstrate that reduction of progranulin recapitulates the pathological proteolysis of TDP-43 observed in FTLD-U and ALS. In addition, caspase-3 was identified as the protease responsible for this cleavage. These results provide insight into the processes underlying diseases with TDP-43 accumulation as a neuropathological feature.

### Example 2

### Using TDP-43 and TDP-43 Cleavage Products to Detect Neurodegenerative Diseases

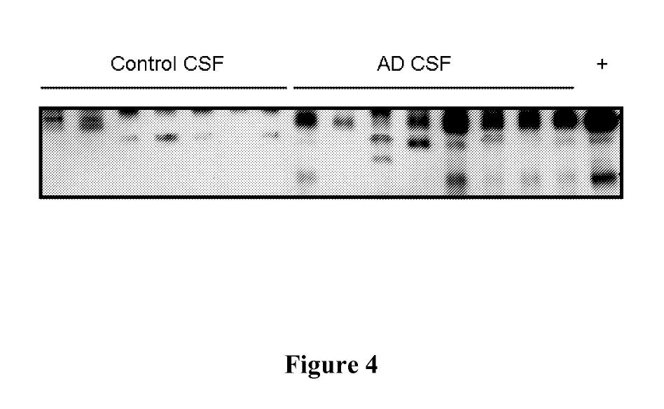

Cerebrospinal fluid samples were obtained from control humans and humans with AD and used to perform a Western blot (25 μg total protein) using a rabbit-anti-TDP-43 antibody. Western blot revealed an increase in total TDP-43 levels (full-length, FIG. 4 “*”) and a truncated TDP-43 (35 kD; FIG. 4 “**”) species in a subset of AD patients. Hela cell line extract treated with staurosporine served as a positive control. These results demonstrate that a TDP-43 polypeptide or a TDP-43 polypeptide cleavage product can be used to identify mammals having a neurodegenerative disease such as AD.

### Example 3

### Proteasome-Induced Toxicity

The following was performed to investigated whether proteasome-induced toxicity was associated with proteolytic processing of TDP-43.

### Cell Culture and Treatment

H4 neuroglioma cells were grown in Opti-Mem plus 10% FBS and 1% pen-strep. Cells were plated in 6-well plates and at 90% confluency treated with 10 μM proteasome inhibitor I (PSI) (EMD Chemicals, Inc. San Diego, Calif.) or 100 μM pan-caspase inhibitor (Z-VAD-FMK) (EMD Chemicals, Inc. San Diego, Calif.) separately or in combination. Twenty-four hours after treatment, the cells were harvested for subsequent Western blot analysis in the Co-IP buffer (50 mM Tris-HCl, pH 7.4, 1 M NaCl, 1% Triton-X-100, 5 mM EDTA) plus 1% SDS, PMSF, and protease and phosphatase inhibitors.

### Western Blot Analysis

Protein concentrations of cells lysates were measured by a standard BCA assay (Pierce, Rockford, Ill.). Then, the samples were heated in Laemmli's buffer, and equal amounts of protein were loaded into 10-well 10% or 4-20% Tris-glycine gels (Novex, San Diego, Calif.). After transfer, blots were blocked with 5% nonfat dry milk in TBST (TPS plus 0.1% Triton X-100) for 1 hour, and then incubated with rabbit polyclonal TDP-43 antibody (1:500; ProteinTech Group, Inc, Chicago, Ill.), rabbit polyclonal caspase-3 antibody (1:1000; Cell Signaling, Beverly, Mass.), HSP70 (1:2000; Stressgen, Ann Arbor, Mich.), GADPH (1:5000; Biodesign International, Kennebunkport, Me.), or mouse monoclonal β-actin antibody (1:5000, Sigma, Saint Louis, Miss.) overnight at 4° C. Membranes were washed three times each for 10 minutes with TBST and then incubated with anti-mouse or anti-rabbit IgG conjugated to horseradish peroxidase (1:2000; Jackson ImmunoResearch, West Grove, Pa.) for 1 hour. Membranes were then washed three times each for 10 minutes, and protein expression was visualized by ECL treatment and exposure to film.

### Results

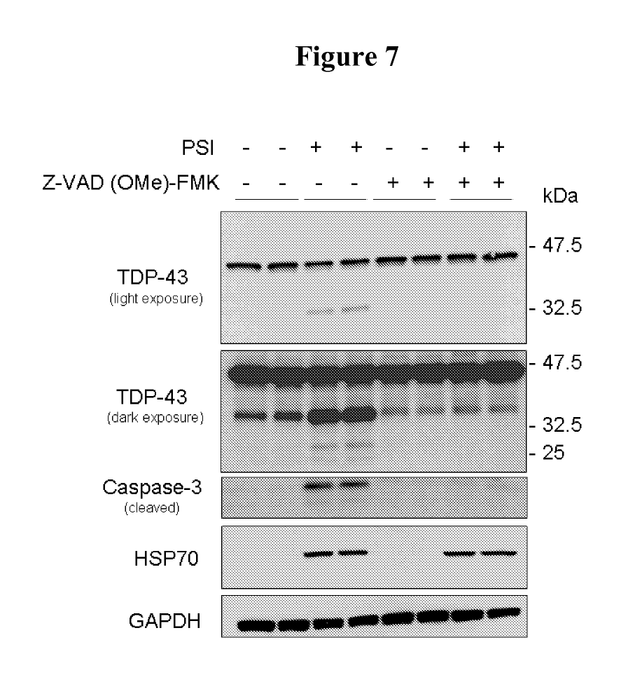

H4 cells were treated with either vehicle (DMSO) or PSI (10 μM) for 24 hours. In the presence of PSI, endogenous cellular TDP-43 was cleaved into ˜35 and ˜25 kD fragments (FIG. 7). A reduction in the full length TDP-43 band (light exposure; ˜43 kD) was observed under these conditions (FIG. 7). The inhibitory activity and toxicity of PSI also led to a marked increase in cleaved (active) capase-3 levels, which promotes apoptotic cell death and accumulates upon such inhibition. Furthermore, when the cells were co-treated with PSI and the caspase inhibitor, Z-VAD (OMe)-FMK, the generation of proteolytic TDP-43 fragments was inhibited (FIG. 7). HSP70 immunoblot analysis was used to verify the inhibition of the proteasomal machinery. As expected, HSP70 levels were increased after PSI treatment, and the levels persisted in the presence of caspase inhibitor Z-VAD (OMe)-FMK (FIG. 7). Taken together, these results demonstrate that proteasome inhibition is sufficient to promote proteolytic cleavage of TDP-43 through a mechanism that implicates programmed cell death.

### Example 4

### Anti-P25 TDP-43 Antibodies

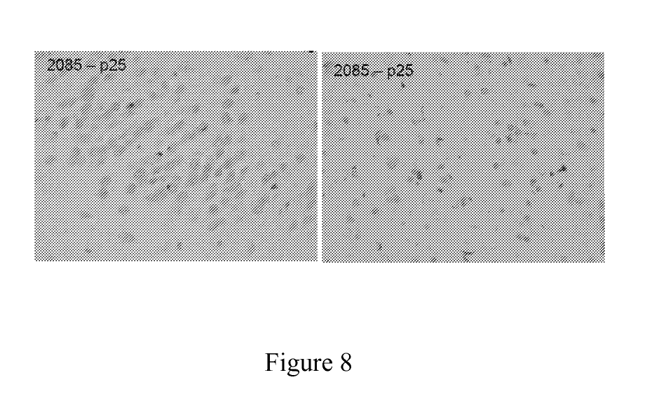

Polyclonal antibodies were made to various TDP-43 polypeptide fragments including VFIPKPFR (SEQ ID NO:3), which is a portion of the ˜25 kD fragment of TDP-43 that follows the caspase cleavage site. The polyclonal antibody raised against VFIPKPFR was designated MC2085 and was discovered to provide disease-specific staining without the normal nuclear staining (FIG. 8). This antibody, raised against the predicted N-terminal polypeptide of the ˜25 kD caspase cleavage product, demonstrated diagnostic capabilities in identifying TDP-43-positive FTLDu cases (FIG. 8). These results demonstrate that the MC2085 antibody and other antibodies that detect the ˜25 kD fragment of TDP-43 can be used to specifically detect pathology in, for example, clinical histological specimen.

### Example 5

### Aberrant Cleavage of TDP-43 Enhances Aggregation and Cellular Toxicity

### Plasmids

The full-length human TDP-43 cDNA in the plasmid pENTR-221 (Invitrogen) was used as the PCR template to generate N-terminal enhanced GFP-tagged TDP-43 plasmids. The N-terminal GFP-tagged cDNA encoding TDP-43, TDP-35, TDP-25, ΔNR1, TDP1-257, and TDP1-175 were generated by PCR by the addition of a GFP tag to the 5′ end of TDP-43. The primers used were: 43-kDa: 5′-CGGGATCCATGTCTG-AATATATTC-3′ (SEQ ID NO:6) and 5′-GCTCTAGACATTCCCCAGCCAGAAG-3′(SEQ ID NO:7); 35-kDa: 5′-CGGGATCCATGGCTTCATCAGCAG-3′ and 5′-GCTCTAGACATTCCCCAGCCAGAAG-3′ (SEQ ID NO:8); 25-kDa: 5′-CGGG-ATCCATGGTCTTCATCCCCAAGC-3′ (SEQ ID NO:9) and 5′-GCTCTAGACAT-TCCCCAGCCAGAAG-3′ (SEQ ID NO:10); ΔNR1: 5′-CGGGATCCGTGTTTGTG-GGGCGCTG-3′ (SEQ ID NO:11) and 5′-GCTCTAGACATTCCCCAGCCA-GAAGAC-3′ (SEQ ID NO:12); 76-414: 5′-CGGGATCCAAACTTCCT-AATTCTAAGC-3′ (SEQ ID NO:13) and 5′-GCTCTAGACATTCCCCAGCCAGA-AGAC-3′ (SEQ ID NO:14); 1-257: 5′-CGGGATCCATGTCTGAATATATTCGGG-3′ (SEQ ID NO:15) and 5′-GCTCTAGATATATGAACGCTGATTC-3′ (SEQ ID NO:16); and 1-175: 5′-CGGGATCCATGTCTGAATATATTCGGG-3′ (SEQ ID NO:17) and 5′-GCTCTAGAGCAGTCACACCATCGTC-3′ (SEQ ID NO:18). The PCR product was subcloned into the pEGFP-C1 vector (Clontech) by using restriction sites BamHI and XbaI.

### Site-Directed Mutagenesis of TDP-43

Site directed mutagenesis was performed using Quikchange kit (Strategene). Wild-type TDP-43-Myc-His plasmid (Neumann et al., Science, 314(5796):130-133 (2006)) was used as a template to create the S409/410A TDP-43 mutation. The primers used for the mutation were: 5′-CTCAAGCATGGATTCTAAGGCTGCTGGCTGGGG-AATGTCTAG-3′ (SEQ ID NO:19) and 5′-CTAGACATTCCCCAGCCAGCAGCCT-TAGAATCCATGCTTGAG-3′ (SEQ ID NO:20). After confirming the mutation by sequence analysis, the S409/410A GFP-TDP-25 mutation was generated as described above.

### Cell Culture and Treatments

HEK293 cells, grown on glass coverslips, were transfected with 0.3 μg of expression vector (GFP, GFP-TDP-43, GFP-TDP-43-caspase-resistant, GFP-TDP-35, GFP-TDP-25, or GFP-TDP-ΔNR1) by using Lipofectamine 2000 (Invitrogen) according to the manufacturer's instructions then subjected to immunofluorescence analysis 48 hours later. To determine if the cytoplasmic inclusion were insoluble, some cells were treated with 0.2% Triton X-100 prior to fixation, in order to remove soluble protein. To determine if the formation of inclusion was aggresome-dependent, some cells were treated with 1 μM nocodazole (Sigma) for 24 hours after 6 hours of transfection. Finally, to examine if inclusions were ubiquitinated, cells were cotransfected with 0.3 μg of a TDP-43 construct and 0.5 μg HA-ubiquitin, then subjected to immunofluorescence analysis 72 hours later.

### Toxicity Assay

M17 neuroblastoma cells, seeded at 2.5×10³ cells per well in 24-well plates, were grown in Neurobasal A/B27 medium containing Glutamax (Invitrogen) and 10 μM retinoic acid (Sigma) to induce differentiation. Seven days later, cells were transfected with 0.2 μg of expression vector for 72 hours. The medium was collected, and a LDH assay (Promega) was used to measure LDH levels as an indicator of toxicity. Then, cells were fixed, and immunofluorescent staining was done using an antibody against activated caspase-3 (1:250; Cell signaling) and the nuclear marker, bisbenzimide (Hoechst 33258).

### Immunofluorescence

The cultured HEK293 and differentiated M17 cells were fixed with 4% paraformaldehyde in phosphate-buffered saline (PBS) at 4° C. for 15 minutes and permeabilized with PBS-0.5% Triton X-100 for 10 minutes. After blocking with 5% BSA for 1 hour at 37° C., the cells were incubated overnight at 4° C. with mouse monoclonal HA antibody (1:1000, Roche Applied Science), rabbit polyclonal activated caspase-3 antibody (1:250, Cell Signaling), rabbit polyclonal anti-pTDP-43 (which detects phosphorylated S409/S410; 1:1000, COSMO BIO CO., LTD), mouse monoclonal Flag antibody (1:1000, Sigma), rabbit polyclonal M2085 (1:1000), or rabbit polyclonal M2079 (1:1000). After washing, cells were incubated with the Alexa 568-conjugated goat anti-mouse IgG secondary antibody (1:1000, Molecular Probes) or anti-rabbit IgG secondary antibody (1:500, Molecular Probes) at 37° C. for 2 hours. Finally, Hoechst 33258 (1 μg/mL, Invitrogen) was used to stain the nuclei. Images were obtained on a Zeiss LSM 510 META confocal microscope.

### Three-Dimensional Modeling of RNA Recognition Motifs

A portion of TDP-43 (amino acids 101-264) was entered in the LOOPP program (available at on the internet at “cbsuapps” dot “tc” dot “cornell” dot “edu” slash “loopp” dot “aspx”; see also, Arai et al., Biochem. Biophys. Res. Commun., 351(3):602-611 (2006)). The backbone model with the greatest predictive score, 2CGK_A, was used as a template for 3D-modelling. Out of the five ‘hits’ generated, the structure with minimum backbone RMSD (root mean square deviation) (5.34 Å) was chosen. The structure was visualized and plotted using MOLMOL.

### Generation of TDP-43 Antibodies

The polyclonal N-terminal antibody (M2079) and the C-terminal fragment-specific antibody (M2085) were produced by using synthetic peptides to immunize rabbits. The N-terminal antibody (M2079) was raised against the amino acids 2 to 22 of human TDP-43. M2085 was raised against the amino acids 220 to 227, which are C-terminal to the putative caspase cleavage site (DVMD219) within human TDP-43. The synthetic peptides were coupled to KLH and injected into rabbits. The resulting sera were affinity purified and antibody specificity was characterized using Western blot and immunofluorescence analysis.

### Western Blot Analysis

HEK293 cells were grown in 6-well plates in Opti-Mem plus 10% fetal bovine serum and 1% penicillin:streptomycin. When cells reached 90% confluency, they were transfected with 1 μg of each plasmid. After 48 hours, the cells were harvested in lysis buffer (50 mM Tris-HCl, pH 7.4, 1 M NaCl, 1% Triton-X-100, 5 mM EDTA) plus 1% SDS, PMSF, and both a protease and phosphatase inhibitor mixture. Protein concentrations of cells lysates were measured by BCA assay (Thermo Scientific). Samples were prepared in Laemmli's buffer, heated for 5 minutes at 95° C., and equal amounts of protein were loaded into 10-well 10% Tris-glycine gels (Novex). After transfer, blots were blocked with 5% nonfat dry milk in TBST (tris-buffered saline plus 0.1% Triton X-100) for 1 hour, and then the blots were incubated with rabbit polyclonal GFP antibody (1:2000, Invitrogen), rabbit polyclonal anti-pTDP-43 (which detects phosphorylated S409/S410; 1:2000, COSMO BIO CO., LTD), mouse monoclonal GAPDH antibody (1:10000, Biodesign), rabbit polyclonal M2085 (1:200), rabbit polyclonal M2079 (1:200), or mouse monoclonal Flag antibody (1:1000, Sigma) overnight at 4° C. Membranes were washed three times for 10 minutes in TBST then incubated with donkey anti-rabbit or anti-mouse IgG conjugated to horseradish peroxidase (1:5000; Jackson ImmunoResearch) for 1 hour. Membranes were washed three times each for 10 minutes, and protein expression was visualized by ECL treatment and exposure to film.

### Co-Immunoprecipitation

For co-immunoprecipitation studies, HeLa cells were transfected with 1 or 2 μg of each plasmid. Cells were harvested 48 hours later using Co-IP buffer (50 mM Tris-HCl, pH 7.4, 1 M NaCl, 1% Triton X-100, 5 mM EDTA) containing PMSF as well as protease and phosphatase inhibitors. The lysates were sonicated and centrifuged at 16,000 g for 20 minutes. The protein concentration of supernatants was determined by BCA assay (Thermo Scientific). Supernatant containing 500 μg of total protein was combined with Anti-Flag M2 agarose (Sigma) and incubated overnight at 4° C. with gentle shaking. The agarose was pelleted by centrifugation at 2,500 g for 5 minutes and washed with Co-IP buffer 6 times. Captured protein was eluted from the beads using loading buffer and resolved by SDS-PAGE for Western blot analysis.

### Immunohistochemistry

FTLD-U and ALS cases (obtained from Mayo Clinic Jacksonville Brain Bank) were immunostained with either MC2085 or MC2079. Adjacent sections (5 μm thick) from either an ALS or FTLD-U case were deparaffinized and rehydrated in xylene and graded series of alcohol (100, 100, 95 and 70%). Antigen retrieval was performed in dH₂O in steam bath for 30 minutes. The sections were allowed to cool and used for immunohistochemistry on the DAKO Autostainer (DakoCytomation, Carpinteria, Calif., USA) using the DAKO EnVision HRP system. DAKO Liquid DAB Substrate Chromogen system was the chromogen. The slides were then dehydrated and coverslipped.

### RNA Extraction and Semiquantitative RT-PCR

The CFTR mini-gene splicing assay was conducted as described elsewhere (Pagani et al., J. Biol. Chem., 275(28):21041-21047 (2000) and Ayala et al., FEBS Lett., 580(5):1339-1344 (2006)). In brief, Hela cells were co-transfected with 1 μg of minigene construct and 1 μg of GFP-fusion protein expression vector. After 48 hours, cells were harvested and total RNA was extracted with the RNeasy Plus Mini Kit (Qiagen) as suggested by the manufacturer's protocol. Then, 2 μg of total RNA was used to synthesize cDNA using the SuperScript III First-Strand Synthesis System (Invitrogen). For the PCR, 2 μL of cDNA was used in a 20 μL of reaction according to the manufacturer's protocol for Taq PCR Core Kit (Qiagen). The amplification conditions consisted of an initial denaturation step at 94° C. for 3 minutes, followed by 30 cycles of 94° C. for 30 seconds, 62° C. for 60 seconds, and 72° C. for 90 seconds.

### Results

### C-Terminal Fragments of TDP-43 Corresponding to Caspase-Cleavage Products Form Cytoplasmic, Ubiquitin-Positive Inclusions

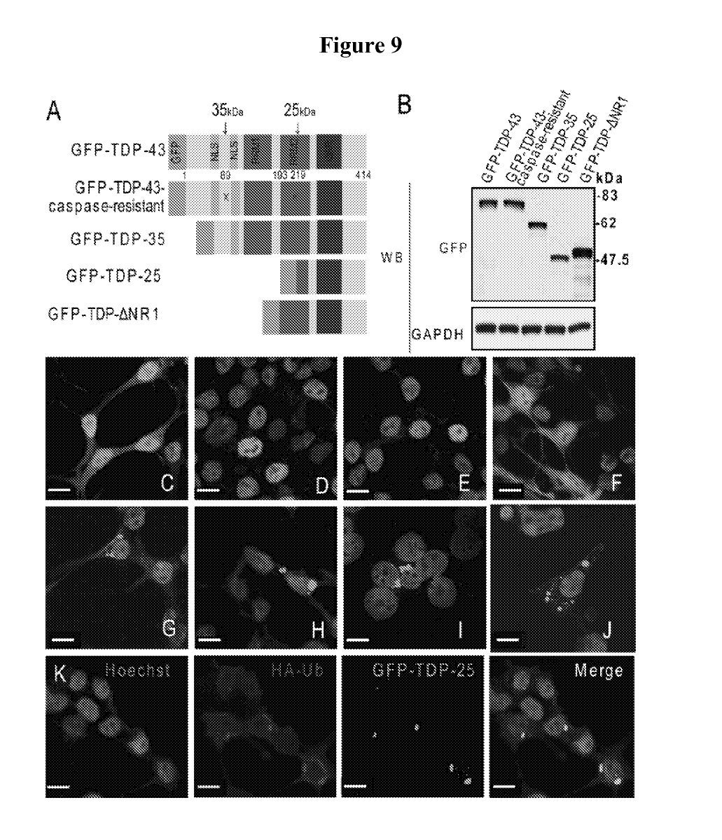

To elucidate the involvement of the C-terminal fragments of TDP-43 in inclusion formation, HEK293 cells were made to express a 35 kDa (aa 90-414) or 25 kDa (aa 220-414) TDP-43 fragment, corresponding to the C-terminal truncation products generated when TDP-43 is cleaved by caspases at residues 89 and 219, respectively (FIG. 9A). To this end, expression vectors were generated encoding the 35 or 25 kDa C-terminal fragments tagged at the amino terminal with enhanced green fluorescence protein (GFP-TDP-35, GFP-TDP-25). Also made were expression vectors for a third C-terminal fragment (aa 193-414) lacking the N-terminus and the RRMI (GFP-TDP-ΔNR1), full-length wild-type TDP-43 (GFP-TDP-43), and caspase-resistant TDP-43 in which the aspartic acid at positions 89 and 219 was substituted to glutamic acid (GFP-TDP-43-caspase-resistant) (Zhang et al., J. Neurosci., 27(39):10530-10534 (2007)) (FIG. 9A). The transient expression of all fusion proteins in HEK293 cells resulted in a similar level of protein expression (FIG. 9B). The cellular distribution of the GFP control, examined by confocal fluorescence microscopy, revealed diffuse cytoplasmic and nuclear distribution (FIG. 9C), while wild-type GFP-TDP-43 (FIG. 9D) and the caspase-resistant mutant (FIG. 9E) localized almost exclusively to the nucleus. In contrast, both GFP-TDP-35 (FIG. 9G) and GFP-TDP-25 (FIG. 9H) exhibited cytoplasmic and nuclear distribution. Moreover, both of these fragments formed compact cytoplasmic inclusions (FIGS. 9G and 9H) with more inclusions observed in cells expressing GFP-TDP-25 (˜75% of cells) compared to GFP-TDP-35 (˜25% of cells). Inclusion formation was specific to caspase-cleavage TDP-43 products, since the GFP-TDP-ΔNR1 C-terminal fragment exhibited diffuse cytoplasmic and nuclear immunoreactivity with no detectable inclusions (FIG. 9F). Of interest, the inclusions formed by GFP-TDP-25 and GFP-TDP-35 were not disrupted by Triton X-100 (FIG. 9I) nor by the microtubule-destabilization agent, nocodazole (FIG. 9J). Additionally, the inclusions were positive for endogenous ubiquitin, and the coexpression of GFP-TDP-25 and HA-tagged ubiquitin demonstrated colocalization of the HA-ubiquitin and GFP within cytoplasmic inclusions (FIG. 9K). Similar results were observed in primary hippocampal neurons and 7 cell lines, including differentiated M17 neuroblastoma cells (FIG. 10), and also when Flag-tagged-TDP-43, -TDP-35, and -caspase-resistant constructs were expressed.

### The 25 KDa C-Terminal Fragment of TDP-43 is Prone to Phosphorylation at S409/S410 but its Phosphorylation is not Required for Inclusion Formation

It has been reported that the TDP-43 C-terminal fragments in the brain of patients with FTLD-U and ALS are phosphorylated at serine 409 and serine 410 (Hasegawa et al., Ann. Neurol., 64(1):60-70 (2008)). To determine if phosphorylation at these sites is required for inclusion formation, HEK293 cells were transfected with GFP-TDP-43 or GFP-TDP-25 and immunostained with a polyclonal antibody specific to TDP-43 when phosphorylated at S409/S410 (anti-pTDP-43). Fluorescent confocal microscopy revealed that the inclusions in cells expressing GFP-TDP-25 were intensely stained by anti-pTDP-43, whereas GFP-TDP-43 was not (FIG. 10A). In a similar fashion, Western blot analysis using extracts from transfected cells showed that, compared to GFP-TDP-43, GFP-TDP-25 was strongly immunoreactive for anti-pTDP-43 (FIG. 10B). In contrast, anti-pTDP-43 failed to detect a mutant TDP-25 incapable of being phosphorylated (GFP-TDP-25S409A/S410A), confirming the specificity of the antibody (FIG. 10B). One possible reason for the enhanced phosphorylation of GFP-TDP-25 is that its cytoplasmic localization renders it readily available to kinases. To establish if full-length TDP-43 would be comparably phosphorylated if it too were present in the cytoplasm instead of the nucleus, an expression vector encoding full-length TDP-43 containing mutations (K82A,R83A,K84A) in the NLS was generated (GFP-TDP-43NLSmut). Substituting the charged amino acid (lysine or arginine) to alanine results in cytoplasmic expression (Ayala et al., J. Cell. Sci., 121(Pt 22):3778-3785 (2008)). Cells transfected with GFP-TDP-43NLSmut had the same modest anti-pTDP-43 immunoreactivity as GFP-TDP-43 (FIG. 10B), suggesting that cytoplasmic distribution does not account for the enhanced phosphorylation of GFP-TDP-25. Finally, since GFP-TDP-25 expression causes inclusion formation and since it is heavily phosphorylated, the following was performed to determine if preventing phosphorylation of GFP-TDP-25 at S409/S410 would prevent inclusion formation. Thus, cells expressing GFP-TDP-25S409A/S410A were examined by fluorescent confocal microscopy. As shown in FIG. 10C, despite the inability to be phosphorylated, GFP-TDP-25S409A/S410A formed cytoplasmic inclusions similar to those formed by GFP-TDP-25 indicating that phosphorylation at S409/S410 is not required for TDP-25 aggregation. Together, these results suggest that the 25 kDa C-terminal fragment of TDP-43 is more prone to phosphorylation at S409/S410 than full-length TDP-43, but enhanced phosphorylation is not required for its aggregation into inclusions.

### The 25 KDa C-Terminal Fragment of TDP-43 Causes Cellular Toxicity

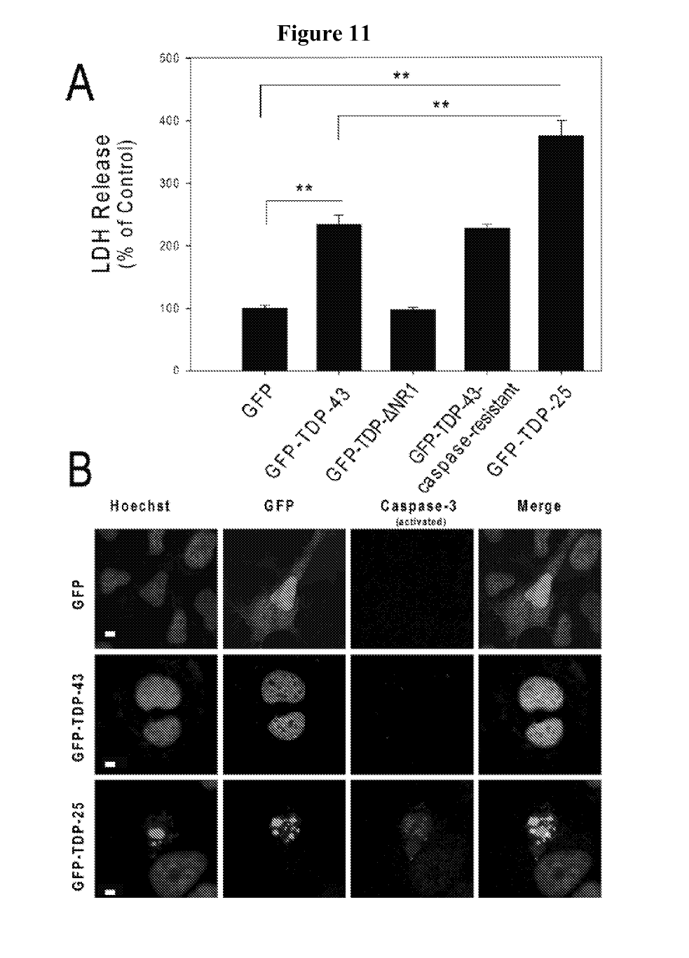

The following was performed to determine if GFP-TDP-25 cytoplasmic aggregates increase cellular toxicity. Differentiated M17 neuroblastoma cells were transiently transfected with the GFP, GFP-TDP-43, GFP-TDP-43-caspase-resistant, GFP-TDP-ΔNR1, or GFP-TDP-25 vector. The medium was collected 72 hours later and lactate dehydrogenase (LDH) levels were measured. Compared to LDH levels in cells expressing only GFP, LDH levels were no different in GFP-TDP-ΔNR1-expressing cells, but were comparably increased in GFP-TDP-43 and GFP-TDP-43-caspase-resistant-expressing cells. GFP-TDP-43 toxicity is not likely due to the presence of the GFP-tag since non-tagged TDP-43, as well as Flag- or Myc-tagged TDP-43, cause the same magnitude of death. Of particular interest, LDH release was significantly higher in cells expressing the 25 kDa C-terminal fragment (FIG. 11A) and GFP-TDP-25S409A/S410A compared to cells transfected with full-length or caspase-resistant TDP-43.

To examine transfection efficiency and the extent of apoptosis, fixed cells were stained with bisbenzimide (henceforth referred to as Hoechst) and an antibody against activated caspsase-3, and then viewed by confocal microscopy. Hoechst labeling of nuclei revealed numerous fragmented nuclei, indicative of apoptotic cell death, only in cells expressing GFP-TDP-25-positive inclusions (FIG. 11B). These inclusions were also immunopositive for activated caspase-3 (FIG. 11B). Based on quantitative analysis, ˜50% of the inclusion-positive cells were positive for fragmented nuclei and activated caspase-3. In contrast, cells expressing GFP-alone, GFP-TDP-43 (FIG. 11B) or GFP-TDP-43-caspase-resistant did not stain for activated caspase-3. These results suggest that GFP-TDP-25 expression and inclusion formation is deleterious to differentiated neuroblastoma cells.

The 25 KDa C-Terminal Fragment of TDP-43 does not Sequester Full-Length TDP-43

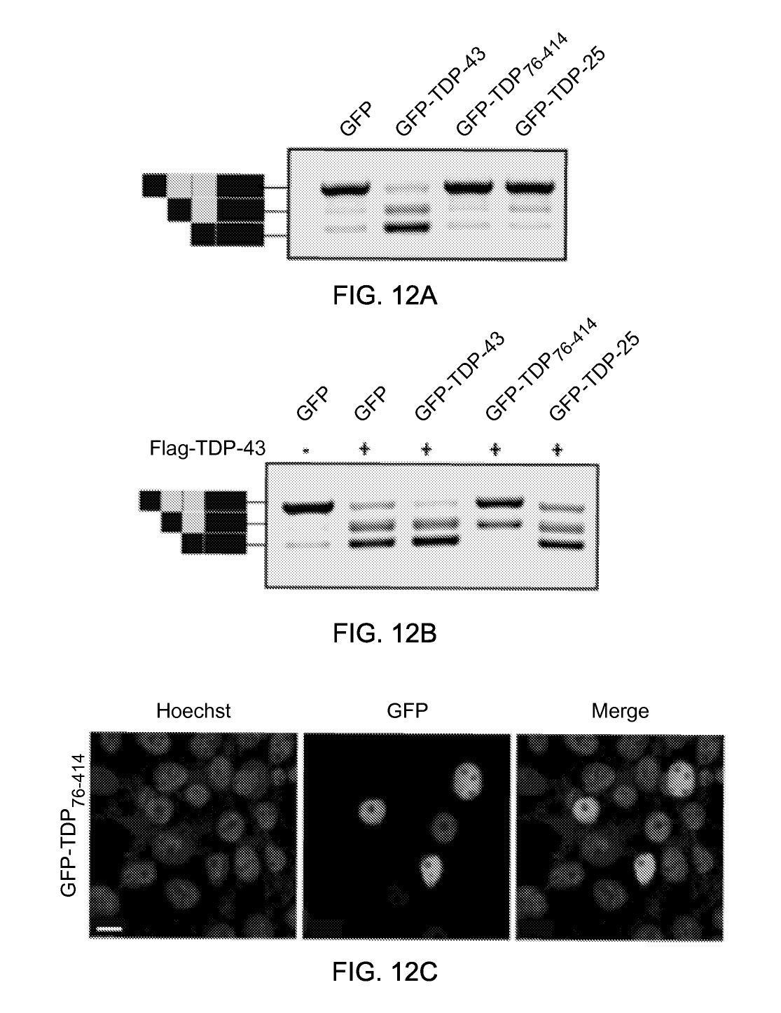

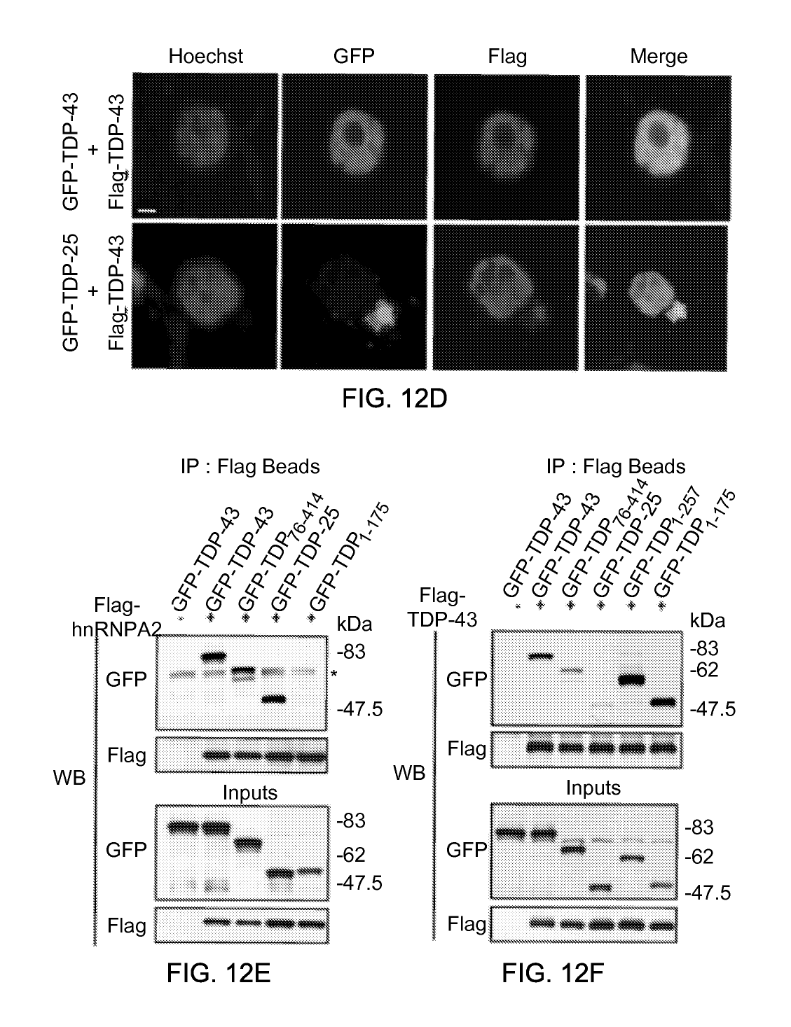

The toxicity associated with the expression of the 25 kDa C-terminal fragment may result from a toxic gain-of-function due to its abnormal presence in the cytoplasm, or from the sequestration of endogenous wild-type TDP-43. If the 25 kDa fragments lead to depletion of endogenous nuclear TDP-43, a decrease in the activity of TDP-43 is anticipated. One of the biological activities of TDP-43 is promoting the skipping of cystic fibrosis transmembrane conductance regulator (CFTR) exon 9 (Buratti and Baralle, J. Biol. Chem., 276(39):36337-36343 (2001)). To examine if the 25 kDa fragment would reduce this activity, HeLa cells were cotransfected with an expression vector for GFP-TDP-25, GFP-TDP-43, or GFP, and a CFTR minigene construct, previously shown to mimic the splicing pattern of endogenous exon 9 CFTR (Pagani et al., J. Biol. Chem., 275(28):21041-21047 (2000)) (FIG. 12A-B). Additionally, since little is known about the normal biological function of the N-terminal of TDP-43, except that it contains the nuclear localization sequence, the effect of a second C-terminal fragment was examined: GFP-TDP76-414. Unlike GFP-TDP-25, this fragment retains nuclear distribution (FIG. 12C), contains all the critical functional domains (FIG. 9A) and does not increase toxicity. Following cotransfection, the relative mRNA levels of exon inclusion and exclusion products were monitored by RT-PCR. In cells cotransfected with GFP, little exon exclusion activity was present, suggesting that endogenous TDP-43 activity is low, especially when compared to the marked increase in exon exclusion observed with GFP-TDP-43 expression (FIG. 12A). If GFP-TDP-25 causes toxicity by depleting endogenous nuclear TDP-43, a decrease in TDP-43 activity is expected. However, the exon inclusion/exclusion profiles did not differ between cells expressing only GFP and those expressing GFP-TDP-25 (FIG. 12A). This suggests that GFP-TDP-25 has no effect on endogenous TDP-43 activity, perhaps because it lacks an N-terminal region required for TDP-43 sequestration. Interestingly, despite its nuclear distribution (FIG. 12C), GFP-TDP76-414 did not promote exon 9 exclusion indicating the importance of the N-terminal domain for normal TDP-43 activity (FIG. 12A).

The results above suggest that the 25 kDa C-terminal fragment has no effect on endogenous TDP-43 activity. Because endogenous TDP-43 exon skipping activity was low (FIG. 12A), any reduction caused by GFP-TDP-25 could be difficult to accurately detect. Therefore, the effect of the 25 kDa C-terminal fragment on the exon skipping activity in cells overexpressing full-length TDP-43 was examined. To this end, cells were transfected with the CFTR mini-gene construct and Flag-TDP-43, as well as either the GFP-TDP-25, GFP-TDP-43, GFP-TDP76-414, or GFP vector. The co-expression of Flag-TDP-43 and GFP-TDP-43 promoted exon 9 exclusion (FIG. 12B). In contrast, the expression of GFP-TDP-25 with Flag-TDP-43 did not reduce Flag-TDP-43 activity (FIG. 12B). Of interest, the expression of GFP-TDP76-414 markedly reduced the exon skipping activity of Flag-TDP-43 (FIG. 12B).

Based on these results, it seems unlikely that the 25 kDa C-terminal fragment causes toxicity by sequestering full-length TDP-43 in the cytoplasm and preventing its nuclear function. This is further supported by confocal fluorescent microscopy experiments in which HEK293 cells were co-transfected with Flag-TDP-43 and GFP-TDP-25 or GFP-TDP-43. As expected, anti-Flag immunoreactivity and GFP fluorescence co-localized in the nucleus of cells co-transfected with Flag-TDP-43 and GFP-TDP-43 (FIG. 12D). In cells expressing both Flag-TDP-43 and GFP-TDP-25, immunostaining with an anti-Flag antibody strongly stained nuclear Flag-TDP-43 but exhibited only weak cytoplasmic staining. Thus, the nuclear localization of Flag-TDP-43 was not appreciably altered by GFP-TDP-25, further indicating that this fragment does not sequester nuclear TDP-43 to the cytoplasm.

Though the 25 kDa C-terminal fragment did not affect TDP-43 activity, the nuclear C-terminal fragment, GFP-TDP76-414, inhibited the activity of TDP-43 (FIG. 12B). This result was surprising since it was presumed that GFP-TDP76-414 would enhance activity because it contains all the functional domains similar to wild-type TDP-43 and is present in the nucleus. In an effort to determine why GFP-TDP76-414 had little activity compared to GFP-TDP-43, its ability to bind hnRNPA2, an interaction that is likely essential for the splicing inhibitory activity of TDP-43 (Buratti et al., J. Biol. Chem., 280(45):37572-37584 (2005)), was examined. Thus, co-immunoprecipitation experiments were performed in which HeLa cells were cotransfected with Flag-tagged hnRNPA2 and GFP-TDP76-414, GFP-TDP-43, GFP-TDP-25 or a C-terminal deletion mutant (GFP-TDP1-175), used as a negative control. Co-immunoprecipitation of Flag-tagged hnRNPA2 followed by probing for GFP indicated that GFP-TDP76-414 did indeed interact with hnRNPA2, as did GFP-TDP-43 and GFP-TDP-25 (FIG. 12E). GFP-TDP1-175, which lacks the critical glycine rich region necessary for interaction, failed to bind hnRNPA2 (FIG. 12C). Thus, the loss of GFP-TDP76-414 activity is not due to an inability to bind hnRNPA2.

Next, to determine if the inhibitory effect of GFP-TDP76-414 on TDP-43 activity resulted from an interaction between GFP-TDP76-414 and wild-type TDP-43, co-immunoprecipitation experiments were performed. HeLa cells were co-transfected with Flag-TDP-43 and the vector for GFP-TDP76-414, GFP-TDP-43, GFP-TDP-25, GFP, or the C-terminal deletion products (GFP-TDP1-175 and GFP-TDP1-257). Co-immunoprecipitation of Flag-tagged TDP-43 followed by probing for GFP revealed that Flag-TDP-43 bound strongly to GFP-TDP-43 as well as to the C-terminal deletion products, GFP-TDP1-175 and GFP-TDP1-257 (FIG. 12F). In contrast, GFP-TDP76-414 and GFP-TDP-25 only weakly interacted with Flag-TDP-43, despite comparable expression levels (FIG. 12F). Thus, the ability of GFP-TDP76-414 to inhibit Flag-TDP-43 activity does not occur though the interaction of these two proteins. It is noteworthy that these results indicate that the interaction of TDP-43 with other TDP-43 molecules appears enhanced by the presence of an intact N-terminal.

### A Conformation-Dependent TDP-43 Antibody Detects C-Terminal TDP-43 Fragments and Specifically Labels the Pathologic Inclusions in Human TDP-43 Proteinopathies

Since the caspase-derived C-terminal fragments aggregate into cytoplasmic inclusions, it was hypothesized that they may be the major pathological species in FTLD-U and ALS. Previous studies have shown that C-terminal specific TDP-43 antibodies detect TDP-43 pathology in FTLD-U and ALS (Igaz et al., Am. J. Pathol., 173(1):182-194 (2008)); however, current commercially available TDP-43 antibodies detect both normal nuclear TDP-43 as well as TDP-43-positive pathological structures, like neuronal cytoplasmic inclusions (NCI), dystrophic neurites (DN), and neuronal intranuclear inclusions (NII). In some cases, extensive nuclear labeling makes it difficult to detect mild TDP-43 pathology, and antibodies to TDP-43 phospho-peptides appear to be lesion-specific (Hasegawa et al., Ann. Neurol., 64(1):60-70 (2008

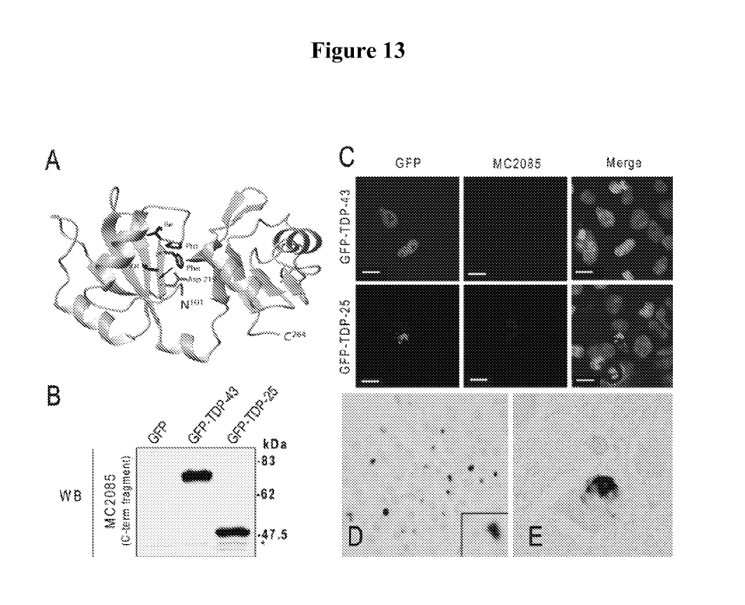

As described herein, a polyclonal TDP-43 antibody (MC2085) was generated to a peptide sequence in the 25 kDa C-terminal fragment. Based on composite analysis of the TDP-43 amino acid sequence and 3-dimensional modeling (FIG. 13A), the amino acid residues, VFIPKPFR, C-terminal to the putative caspase cleavage site, was selected as the peptide antigen.

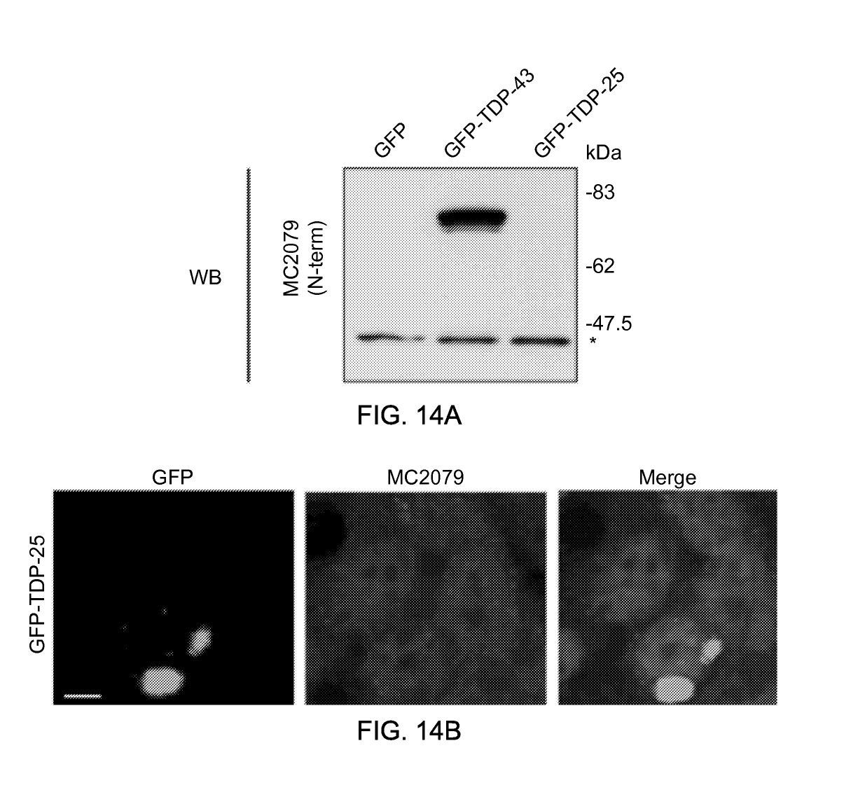

To determine if MC2085 detects the 25 kDa C-terminal fragment, wild-type TDP-43, or both, cells were transfected with GFP, GFP-TDP-43, or GFP-TDP-25 for Western blot analysis and immunofluorescent confocal microscopy. By Western blot analysis, both GFP-TDP-43 and GFP-TDP-25 were immunoreactive for MC2085 (FIG. 10B). In contrast, immunostaining with MC2085 showed it to react only with the inclusions in GFP-TDP-25 transfected cells and not with nuclear TDP-43 in GFP-TDP-43 transfected cells (FIG. 10C). Of interest, the inclusions were not detected by a TDP-43 N-terminal-specific antibody (MC2079), suggesting they are composed mainly of C-terminal fragments (FIG. 14).

Next, immunohistochemistry of FTLD-U and ALS tissue was conducted using MC2085. Neuronal cytoplasmic inclusions in FTLD-U (FIG. 13D) and ALS (FIG. 13E) cases stained positively for MC2085 but minimal or no staining of normal neuronal TDP-43 and glial nuclei was observed (FIGS. 13D and 13E). These findings suggest that some inclusions in both FTLD-U and ALS contain caspase-derived C-terminal fragments of TDP-43.

### Example 6

### Characterization of Polyclonal Anti-pTDP-43 and Anti-cTDP Antibodies by Immunohistochemistry

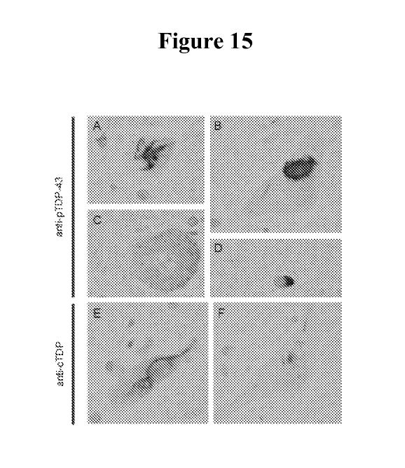

To help establish ELISAs for the measurement of pathologically modified TDP-43, rabbit polyclonal antibodies were generated to detect TDP-43 when phosphorylated at S409/S410 (anti-pTDP-43) or to detect the neoepitope formed upon caspase cleavage of TDP-43 between residues 219 and 220 (anti-cTDP). To determine if the antibodies are lesion-specific, sections of medulla that included the hypoglossal nucleus from ALS cases were immunostained with non-purified anti-pTDP-43 or anti-cTDP. A range of immunoreactive neuronal inclusions were present in the hypoglossal motor neurons in ALS, including skein-like inclusions (FIGS. 15A and 15E) and Lewy body-like inclusions (FIG. 15B). In some neurons, there was no discrete inclusion but rather a redistribution of TDP-43 from the nucleus to punctuate granules in the cytoplasm (FIGS. 15C and 15F). In the tegmentum and inferior olivary nucleus, anti-pTDP-43-immunoreactive glial inclusions were observed (FIG. 15D). The antibodies stain pathologic inclusions with little or no staining of normal nuclei.

### Example 7

### Characterization of Polyclonal Anti-pTDP-43 by Western Blot Analysis

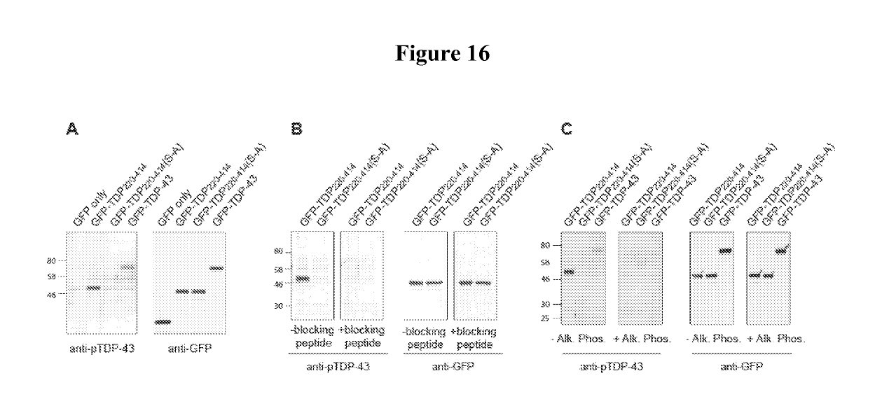

Initial efforts focused on the characterization of anti-pTDP-43. The specificity of non-purified anti-pTDP-43 was tested by Western blot analysis using lysates from HEK293 cells overexpressing full-length human TDP-43 or a C-terminal truncation product corresponding to caspase cleaved TDP-43 (residues 220-414). To this end, cells were transfected with expression vectors encoding wild-type TDP-43 or the 25 kDa fragment tagged at the amino terminal with the enhanced green fluorescence protein (termed GFP-TDP-43 and GFP-TDP₂₂₀₋₄₁₄, respectively). While GFP-TDP-43 and GFP-TDP₂₂₀₋₄₁₄ were both immunoreactive for anti-pTDP-43, the C-terminal fragment was much more prone to phosphorylation (FIGS. 16A and 16C). The staining was specific since incubating anti-pTDP-43 with the immunizing peptide prior to probing blocked its ability to detect GFP-TDP₂₂₀₋₄₁₄ (FIG. 16B). Importantly, anti-pTDP-43 failed to detect mutant GFP-TDP₂₂₀₋₄₁₄ incapable of being phosphorylated due to serine to alanine substitutions at positions 409 and 410 (GFP-TDP_{220-414(S-A)}), validating the specificity of the antibody for phosphorylated S409/S410 (FIGS. 16A-C). This was further confirmed by treating duplicate blots with or without alkaline phosphatase (1 hour, 37° C.) prior to probing with anti-pTDP-43. Dephosphorylated GFP-TDP-43 and GFP-TDP₂₂₀₋₄₁₄ were not immunoreactive for anti-pTDP-43 though immunoreactivity was maintained for the anti-GFP antibody (FIG. 2C).

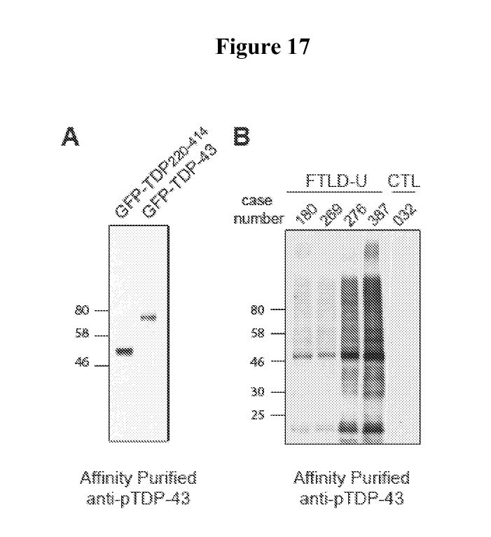

Since affinity purified antibodies are expected to yield better results in an ELISA (e.g., less background), a portion of rabbit serum was used for affinity purification of anti-pTDP-43. The sensitivity of anti-pTDP-43 for GFP-TDP-43 and GFP-TDP₂₂₀₋₄₁₄ was not lost upon purification (FIG. 17A), and it detected phosphorylated TDP-43 present in the urea fraction derived from human brain tissue of FTLD-U, but not control, cases (FIG. 17B).

### Example 8

### Characterization of Polyclonal Anti-pTDP-43 by ELISA

ELISAs are developed using the polyclonal and monoclonal antibodies provided herein for the detection of phosphorylated TDP-43 and C-terminal TDP-43 fragments. To optimize the ELISA conditions, various antibody pairs and concentrations are used. For these experiments, serial dilutions of purified recombinant TDP-43 polypeptides can serve as the antigen. These recombinant TDP-43 polypeptides can serve as standards when using the ELISAs to quantify TDP-43 in biological fluids from humans.

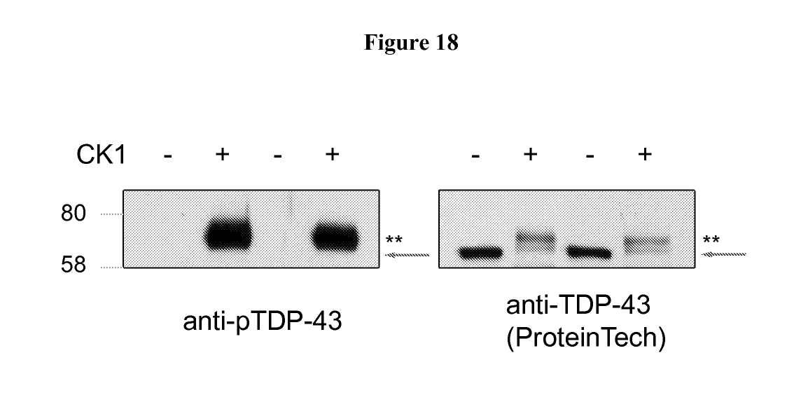

For the detection of phosphorylated TDP-43, recombinant TDP-43 is subjected to in vitro phosphorylation using CK1. Anti-pTDP-43 immunoreactivity of GST-TDP-43 following in vitro phosphorylated by recombinant CK1 (14 hours, 37° C.) is depicted in FIG. 18. Non-phosphorylated GST-TDP-43 was not detected by anti-pTDP-43 (FIG. 18). In some cases, non-tagged, purified, recombinant TDP-43 polypeptides are used as standards. The results provided herein indicate that TDP-43 can be effectively phosphorylated in vitro. Moreover, the results provided herein demonstrate that anti-pTDP-43 can be used to detect phosphorylated GST-TDP-43 by ELISA.

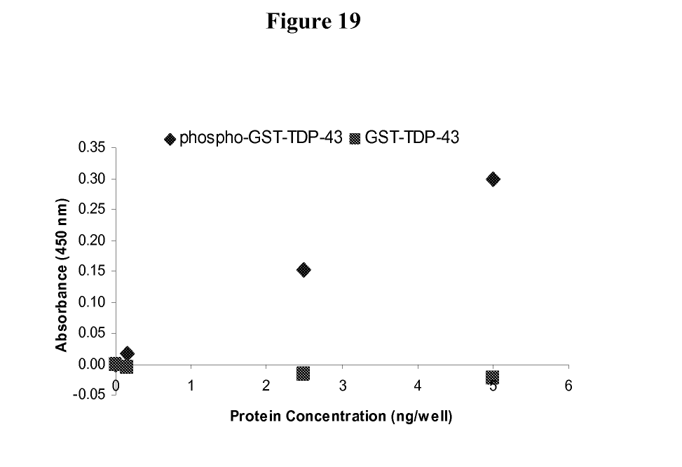

A sandwich ELISA was performed using anti-pTDP-43 (5 μg/mL) as the capture antibody, recombinant GST-TDP-43 that had or had not been subjected to CK1 phosphorylation as the antigen, and a mouse monoclonal antibody (2E2-D3, Novus; 5 μg/mL) as the detection antibody. As shown in FIG. 19, phospho-GST-TDP-43, but not non-phospho-GST-TDP-43, was detected in a concentration-dependent manner. This initial test provides indicates that anti-pTDP-43 can be used in the development of an ELISA to detect pathologically phosphorylated TDP-43.

### Example 9

### Characterization of Monoclonal Phospho-TDP-43 Antibodies

To develop sandwich ELISAs for the detection of phosphorylated TDP-43, a polyclonal pS409/pS410-TDP-43-specific antibody (anti-pTDP-43) was generated by immunizing a rabbit with the peptide, CSMDSK[pS][pS]GWGM-COOH, representing residues 404-414 of human TDP-43. The results of the characterization of anti-pTDP-43 are provided herein (see, e.g., FIGS. 15-19). Given these results, the following was performed to produce and purify monoclonal antibodies against pS409/pS410-TDP-43. Briefly, mice were immunized with the same antigen as was used for the production of the polyclonal anti-pTDP-43. Following immunization, mouse sera were tested for positive response by ELISA. Based on titers to the immunogen, two mice were selected for spleen harvest and the subsequent fusion of spleen cells with mouse myeloma cells. The resulting fusion products were screened by ELISA using the phospho-peptide antigen as the positive control. As a negative control, the same polypeptide sequence was used except that S409 and S410 were not phosphorylated. Eight fusion products exhibiting high reactivity towards the phospho-polypeptide, but not the non-phospho-polypeptide, were selected for subcloning, and 21 additional lines were expanded and frozen. A total of 84 hybridoma lines were obtained for characterization (including parental, daughter and grand-daughter clones).

### Immunohistochemical Studies Using Human Brain Tissue

The supernatants from all 84 clones/subclones are tested immuhistochemically using human brain or spinal cord tissue from control, FTLD-U, and/or ALS patients, as shown in FIG. 15. Initially, tissue is immunostained with neat supernatant from each hybridoma line. The 10 best anti-pTDP-43-producing lines are selected, and serial dilutions of their supernatants are tested to optimize staining conditions and to determine which lines produce the most sensitive and specific antibodies. Those antibodies that stain inclusions and show no nuclear immunoreactivity are considered specific.

### Western Blot Analysis of Anti-pTDP-43 Monoclonal Antibodies

Customary methods are employed to produce and purify recombinant TDP-43 polypeptides. Next, the recombinant TDP-43 polypeptides are phosphorylated in vitro (37° C., 14 hours) by recombinant CK1 (New England BioLabs, Inc) as shown in FIG. 18. The product of this reaction is used to screen for anti-pTDP-43-producing hybridoma cell lines. As the supernatant of 84 clones/subclones is to be tested, the Mini-PROTEAN II Multiscreen Apparatus (Bio-Rad), which is designed to quickly and efficiently screen up to 40 different antibodies via Western blot without having to cut the blot into individual strips for probing, is utilized. Non-phospho-GST-TDP-43 serves as a negative control. The 10 most promising antibodies are next screened using cell lysates from HEK293 cells overexpressing full-length GFP-TDP-43, GFP-TDP₂₂₀₋₄₁₄, or GFP-TDP_{220-414(S-A)}. As shown for the polyclonal anti-pTDP-43 antibody (FIGS. 16 and 17), monoclonal anti-pTDP-43 antibodies are expected to detect GFP-TDP₂₂₀₋₄₁₄ to a much greater extent than GFP-TDP-43, and are not to detect GFP-TDP_{220-414(S-A)}. To further assess if the immunoreactivity of the monoclonal antibodies is specific, antibodies are incubated with the immunizing polypeptide prior to probing, as shown in FIG. 16B for the polyclonal antibody. Additionally, duplicate blots are treated with or without alkaline phosphatase (1 hour, 37° C.) prior to probing, as in FIG. 16C for the polyclonal antibody.

### Example 10

### Characterization of Polyclonal and Monoclonal Anti-cTDP-43 Antibodies

To generate a neoepitope antibody against the caspase cleavage site in TDP-43 that generates a ˜25 kDa C-terminal fragment, the amino acid residues, VFIPKPFR, were used as the polypeptide antigen. Both rabbits and mice were immunized with the same antigen to produce polyclonal and monoclonal antibodies against caspase-cleaved TDP-43, respectively. As for the generation of anti-pTDP-43 monoclonal antibodies, standard monoclonal antibody generation techniques are used.

As demonstrated herein, the polyclonal anti-cTDP-43 detects skein-like inclusions (FIG. 15E) and cytoplasmic punctuate granules (FIG. 15F) in ALS hypoglossal motor neurons. The mouse monoclonal antibodies are to provide similar results.

While polyclonal and monoclonal anti-cTDP-43 antibodies can be designed to specifically detect caspase-cleaved TDP-43, it is possible that these antibodies may act as conformation-dependent antibodies capable of detecting other C-terminal fragments. This would occur if the production of a TDP-43 fragment (due to proteolytic cleavage) unmasks the antibody-binding epitope.

To identify which hybridoma lines produce antibodies towards the neo-epitope of caspase-cleaved TDP-43, ELISAs are performed using the immunizing polypeptide as a positive-control and a polypeptide spanning the caspase-cleavage site as a negative-control. Clones that exhibit positive reactivity against the immunizing polypeptide but not the spanning polypeptide are likely to produce antibodies that specifically detect caspase-cleaved TDP-43. Clones generating antibodies that detect both the immunizing polypeptide and the spanning polypeptide may be useful for the detection of various C-terminal fragments. Both sets of clones are subjected to additional screening assays, as described below. In the case of the polyclonal anti-cTDP antibody, it may be necessary to immunodeplete the antibody using a polypeptide that spans the caspase-cleavage consensus site to eliminate antibodies that detect sites other than the neoepitope.

### Immunofluorescence Staining to Assess Monoclonal and Polyclonal Antibody Specificity

To determine if the polyclonal anti-cTDP-43 antibody and the monoclonal antibodies from generated hybridoma cell lines specifically detect caspase-cleaved TDP-43, HEK293 cells are transfected with various TDP-43 constructs C-terminally tagged with enhanced GFP, including full-length TDP-43 (TDP-43-GFP), caspase-cleaved TDP-43 (TDP₂₂₀₋₄₁₄-GFP), or other TDP-43 fragments (e.g. TDP₂₁₉₋₄₁₄-GFP, TDP₂₀₈₋₄₁₄-GFP and TDP₂₄₇₋₄₁₄-GFP). Two days post-transfection, immunofluorescence studies are carried out. Briefly, cells are fixed, permeabilized, and incubated with the anti-cTDP-43 antibodies. After washing, cells are incubated with the Alexa 568-conjugated goat anti-mouse IgG secondary antibody (1:1000, Molecular Probes) or anti-rabbit IgG secondary antibody (1:500, Molecular Probes). Finally, Hoechst 33258 (1 μg/mL, Invitrogen) is used to stain the nuclei of cells, and images are obtained on a Zeiss LSM 510 META confocal microscope. Antibodies that specifically detect the neo-epitope created upon caspase-cleavage of TDP-43 are expected to detect only TDP₂₂₀₋₄₁₄ but not full-length TDP-43 or other C-terminal fragments. These are termed anti-TDP₂₂₀₋₄₁₄ antibodies. Antibodies that detect C-terminal fragments in a conformation-dependent manner are immunopositive towards various C-terminal fragments, like TDP₂₂₀₋₄₁₄, TDP₂₁₉₋₄₁₄, and/or TDP₂₀₈₋₄₁₄, but will not detect full-length TDP-43. These remain termed anti-cTDP antibodies. Antibodies that detect full-length nuclear TDP-43 are considered conformation- and truncation-independent TDP-43 antibodies. As a second approach to assessing the specificity of anti-TDP₂₂₀₋₄₁₄ antibodies, H4 cells are transfected with either myc-tagged wild-type, full-length TDP-43 or with myc-tagged mutant TDP-43 (D219E) in which the consensus motif for caspase cleavage is mutated. Cells are transfected in the presence or absence of staurosporine, which leads to the activation of caspase 3, and immunofluorescence studies are carried out as above.

Wild-type full-length myc-TDP-43 localizes to the nucleus of H4 cells but, upon staurosporine treatment and TDP-43 cleavage, TDP-43 redistributes to the cytoplasm. In contrast, caspase mutant TDP-43 retains its nuclear localization even after staurosporine treatment. If anti-TDP₂₂₀₋₄₁₄ antibodies specifically detect caspase-cleaved TDP-43, then these antibodies are expected to only detect cytoplasmic TDP-43 following staurosporine treatment of wild-type full-length myc-TDP-43 transfected cells and not to detect caspase-mutant TDP-43.

### Immunohistochemical Studies Using Human Brain Tissue

The polyclonal anti-cTDP-43 was tested on human tissue (FIG. 15). Similarly, the supernatants from all anti-cTDP or anti-TD₂₂₀₋₄₁₄-producing hybridoma lines are tested immunohistochemically using human brain tissue from control, FTLD-U, and/or ALS patients to determine if anti-cTDP and anti-TDP₂₂₀₋₄₁₄ antibodies are lesion-specific.

### Western Blot Analysis of Anti-cTDP and Anti-TDP₂₂₀₋₄₁₄ Antibodies

To determine if the antibodies can detect TDP-43 by Western blot, Western blotting assays are conducted using lysates from HEK293 or H4 cells transfected with the full-length and cleaved TDP-43 constructs or using purified recombinant full-length and truncated (residues 220-414) TDP-43. Additionally, lysates are prepared from human ALS, FTLD-U, and control brain tissue using a sequential extraction method to obtain a sarkosyl-soluble fraction and a sarkosyl-insoluble/urea soluble fraction. Since anti-TDP₂₂₀₋₄₁₄ antibodies are designed to detect TDP-43 following caspase cleavage or under denaturing conditions, it is expected that one or more of the antibodies may detect full-length TDP-43 in addition to TDP₂₂₀₋₄₁₄ by Western blot, even though they fail to detect full-length TDP-43 during the immunofluorescence studies detailed above. The same is expected for the confirmation-dependent pan-C-terminal anti-cTDP antibodies. To confirm antibody specificity, blocking experiments are performed using the immunizing polypeptide as a blocking polypeptide, as shown in FIG. 16.

### Example 11

### Establishing Optimal ELISA Conditions for the Detection of pS409/pS410-TDP-43 and C-Terminal TDP-43 Fragments

Using the above-characterized antibodies, sandwich ELISAs are established for the selective detection of phosphorylated (S409/S410), caspase-cleaved ˜25 kDa TDP-43, and C-terminal TDP-43 fragments. ELISAs that detect TDP-43 independently of these modifications are also developed. Initially, optimal ELISA conditions are determined by testing different coating buffers (e.g., phosphate-buffered saline versus sodium carbonate buffer), capture/detection antibody concentrations as well as capture/detection antibody pairs (Table 1). For these experiments, serial dilutions of purified recombinant TDP-43 polypeptides serves as the antigen. These recombinant TDP-43 polypeptides later serve as standards when using the ELISAs to quantify TDP-43 in biological samples from humans. Customary methods are employed to produce and purify the recombinant TDP-43 polypeptides.

Recombinant truncated (aa 220-414) or full-length (aa 1-414) TDP-43 is used to determine the optimal ELISA conditions for the detection of C-terminally cleaved or full-length TDP-43, respectively. Non-tagged recombinant TDP-43 or purified GST-tagged TDP-43 is used as a standard (see, e.g., FIGS. 18 and 19). For the detection of phosphorylated TDP-43, recombinant TDP-43 is subjected to in vitro phosphorylation using CK1. As indicated herein, CK1 effectively phosphorylated GST-TDP-43 at S409/S410 in vitro (FIG. 18). Once generated, monoclonal antibodies to specifically detect TDP-43 when not phosphorylated at the S409/S410 sites, together with anti-pTDP-43 antibodies, are used to determine the efficiency of in vitro phosphorylation.

TABLE 1
Antibody pairs to test for the detection of pathologically modified and total TDP-43.

| | Antibody Pairs | Recombinant TDP-43 |
|---|---|---|
| | (each antibody of a given pair is tested as | (is used to determine optional |
| Detection Of: | both the capture and detection antibody) | ELISA conditions) |
| S409/S410-phosphorylated | mono- or polyclonal anti-pTDP-43 and: | in vitro phosphorylated |
| TDP-43 | 1) 2E2-D3* | recombinant TDP-43 |
| | 2) 10782-2-AP^{&} | |
| | 3) ab54502^{#} | |
| Caspase-cleaved TDP-43 | mono- or polyclonal anti-TDP₂₂₀₋₄₁₄ and: | recombinant truncated TDP- |
| | 1) 10782-2-AP^{&} | 43 (aa 220-414) |
| | 2) ab54502^{#} | |
| | 3) MC2078^{£} | |
| C-terminal TDP-43 | mono- or polyclonal anti-cTDP and: | recombinant truncated TDP- |
| | 1) 10782-2-AP^{&} | 43 (aa 220-414) |
| | 2) ab54502^{#} | |
| | 3) MC2078^{£} | |
| Total TDP-43 | 1) 2E2-D3 and 10782-2-AP^{&} | Recombinant full-length |
| | | TDP-43 |

*2E2-D3: mouse monoclonal antibody that detects residues 205-222 of TDP-43 (Novus)
^{&}10782-2-AP: rabbit polyclonal antibody generated against recombinant protein (1-260) (Protein Tech)
^{#}ab54502: rabbit polyclonal raised against a polypeptide corresponding to residues 350-414 (Abcam)
^{£}MC2078: rabbit polyclonal antibody generated against a polypeptide corresponding to residues 403-414

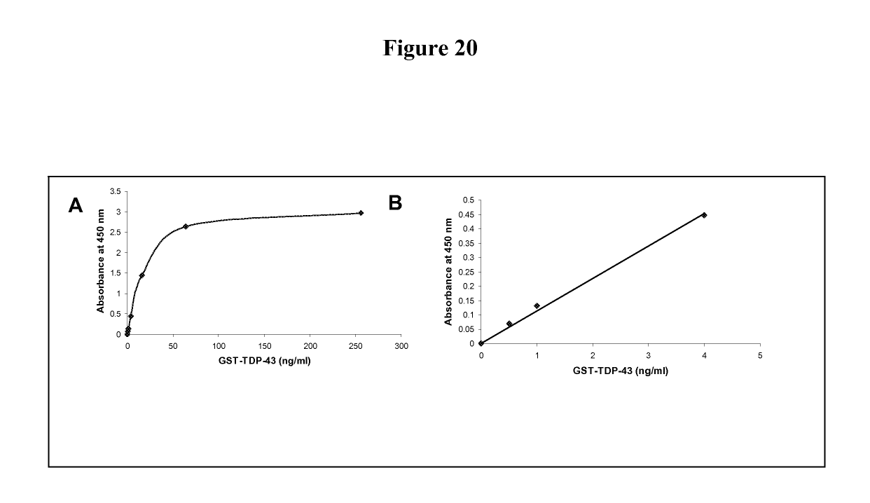

In brief, to test different ELISA conditions, 96-well plates are coated with a capture antibody (0.2-5 μg/mL; 100 μL/well) in either phosphate-buffered saline (PBS) or sodium carbonate buffer, overnight at 4° C. The following day, each well is incubated with 300 μL blocking buffer for 2 hours at 37° C. After washes in PBST (PBS containing 0.05% Tween 20), 100 μL of recombinant protein (0.025-25 ng/well) is added to the wells in duplicate or triplicate and left to incubate overnight at 4° C. Next, the unbound products is removed by washes, and a peroxidase-conjugated detection antibody (0.2-5 μg/mL; 100 μL/well) is added to each well for 2 hours at 37° C. The Peroxidase Labeling Kit (Roche Applied Science) is used to label the detection antibodies with activated peroxidase. After the final washes, the TMB Microwell Peroxidase Substrate System (KPL) is used to measure the amount of peroxidase-labeled detection antibody bound to the antigen. The various capture and detection antibody pairs to be tested are shown in Table 1. For the detection of TDP-43 independently of its phosphorylation or truncation state, the antibodies 2E2-D3 and 10782-2-AP are used as shown in FIG. 20.

Following the optimization of sandwich ELISA conditions, the assays are validated using lysates from HEK293 cells overexpressing full-length TDP-43 or cleaved TDP-43(TDP₂₂₀₋₄₁₄, TDP₂₁₉₋₄₁₄ or TDP₂₀₈₋₄₁₄). As demonstrated herein, TDP₂₂₀₋₄₁₄ is phosphorylated at S409 and S410 (FIG. 16), as are TDP₂₁₉₋₄₁₄ or TDP₂₀₈₋₄₁₄. Therefore, lysates from these cells is used to test the phospho-specific ELISAs as well as the ELISAs generated to detect TDP₂₂₀₋₄₁₄ and C-terminal TDP-43 fragments.

### Example 12

### Determining if Levels of Pathologically Modified TDP-43 in Human Biological Samples Correlate with TDP-43 Pathology as Well as Severity and Rate of Disease Progression

To determine if TDP-43 is a reliable biomarker of TDP-43 proteinopathies, TDP-43 levels are measured in plasma obtained from normal individuals (˜30 samples), AD patients with either histopathologically-confirmed TDP-43-positive (˜10 samples) or TDP-43-negative (˜30 samples) pathology, FTLD-U patients with TDP-43 pathology (˜30 samples) or FTLD patients with expected tau-only pathology (7 samples from patients with corticobasal degeneration or progressive supranuclear palsy). Plasma TDP-43 levels are measured using the ELISAs described herein to detect S409/S410-phosphorylated TDP-43, caspase-cleaved TDP-43, and C-terminal TDP-43 fragments. Additionally, an ELISA that detects TDP-43 independently of its phosphorylation or truncation state are used. Purified full-length, truncated or in vitro phosphorylated recombinant TDP-43 polypeptides are used to generate standard curves. ELISAs are performed in a blinded fashion without knowledge of which subject provided the specimen. After completion of the ELISAs, subject codes are revealed for data analysis.

TDP-43 levels may be highest in patients with confirmed TDP-43 pathology compared to patients with TDP-43-negative, tau-positive pathology, and the levels of phosphorylated and/or truncated TDP-43 may be a more sensitive indicator of TDP-43 pathology than levels of total TDP-43.

To determine if TDP-43 levels in biological fluids can serve as a reliable index of disease progression and severity, TDP-43 levels are measured in plasma, serum, and CSF samples collected longitudinally from ALS patients. The ELISAs described herein are used to measure phosphorylated, caspase-cleaved, N-terminally truncated, and total TDP-43 in plasma, serum, and CSF obtained from 20 ALS patients over the course of 6 months (at baseline, month 3 and month 6) as well as in samples from control subjects.

In brief, aliquots of plasma, serum, and CSF samples are obtained from eligible subjects with ALS, between 21 to 85 years of age. Inclusion criteria include those commonly used in ALS therapeutic trials as this is a population of interest for potential application of biomarkers in clinical research. Similarly, exclusion criteria include criteria used in previous multi-center ALS treatment trials. Eligible individuals with ALS are included without discrimination based on race, ethnicity, or gender. Clinical assessment of the 20 ALS subjects using the revised ALS functional rating scale (ALSFRS-R) is performed monthly, either in person at the baseline, month 3 and month 6 visits, or by telephone at months 1, 2, 4 and 5. The ALSFRS-R is a functional outcome measure used for the assessment of ALS disease progression. A 6-month follow-up interval is anticipated to be long enough to reasonably evaluate ALS progression, given that an analysis of clinical trial design suggests that a 4-month interval with monthly evaluations should reveal evidence of disease progression using the ALSFRS. The Mini-Mental Status Examination (MMSE) of Folstein, Folstein and McHugh is used to screen for cognitive impairment at baseline. As cognitive impairment may be present in as many as 50% of ALS patients, a MMSE score <24 (considered clinically significant in normal subjects) is not an exclusion factor. Up to 20% of patients with ALS demonstrate signs and symptoms of frontal temporal cognitive impairment (FTCI). At baseline, as wells as at months 3 and 6, the ALS Cognitive Behavioral Screen (ALS-CBS) is used to screen for the presence of FTCI, as this feature of ALS may correlate with pathologically modified TDP-43 levels in plasma, serum, and/or CSF.

Blood samples from ALS subjects are collected into standard EDTA vacutainer tubes or serum separator tubes and are centrifuged to collect plasma and serum, respectively. Plasma and serum are maintained on wet ice while aliquoted to labeled cryotubes and then are frozen at −80° C. Subjects with acceptable laboratory results proceed with a standard lumbar puncture by specially trained nurses. CSF designated for TDP-43 ELISAs are kept on wet ice while aliquoted to cryotubes and then are frozen at −80° C. The BARD labeling system is used to de-identify the plasma, serum, and CSF specimens and to assign each specimen a subject-specific code number for tracking and storage purposes. Subject data, including ALSFRS-R and MMSE scores, are entered into an electronic database used for the study. All patient-specific study data is kept confidential.

Serum, plasma, and CSF samples from the ALS and controls subjects are tested in duplicate using the ELISAs described herein to detect S409/S410-phosphorylated TDP-43, caspase-cleaved TDP-43, and C-terminal TDP-43 fragments. Additionally, an ELISA that detects TDP-43 independently of its phosphorylation or truncation state is used. Purified full-length, truncated or in vitro phosphorylated recombinant TDP-43 polypeptides are used to generate standard curves. The data is analyzed to determine if (1) CSF, serum, and/or plasma TDP-43 levels are higher in ALS subjects compared to control subjects, (2) TDP-43 levels change with the evolution of disease, (3) TDP-43 levels correlate with disease severity and/or cognitive impairment, (4) measuring pathologically modified TDP-43 provides a more sensitive diagnostic tool than measuring TDP-43 using phosphorylation- and truncation-independent antibodies, and (5) TDP-43 levels are similar among plasma, serum, and CSF samples.

To examine if levels of normal or pathologically modified TDP-43 are higher in biological samples from ALS subjects than from controls, groups (control samples versus ALS samples at baseline) are compared using the Mann-Whitney U test. To examine if CSF, serum, or plasma TDP-43 levels change over time in ALS, Kendall's correlation coefficient tau is used to examine the degree of association between plasma, serum, or CSF TDP-43 for each separate time-point (baseline, month 3 and month 6) as well as overall. The association of ALSFRS-R scores with plasma, serum, or CSF TDP-43 levels, as well as the association of ALS-CBS with plasma, serum or CSF TDP-43 levels, are evaluated by Kendall's tau.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing a mammal for a neurodegenerative disease, wherein said method comprises determining whether or not a biological fluid from said mammal contains an elevated level of a TDP-43 polypeptide or a TDP-43 polypeptide cleavage product, wherein the presence of said elevated level indicates that said mammal has said neurodegenerative disease.

2. The method of claim 1, wherein said mammal is a human.

3. The method of claim 1, wherein said neurodegenerative disease is frontotemporal dementia, Alzheimer's disease, or amyotrophic lateral sclerosis.

4. The method of claim 1, wherein said biological fluid is a cerebrospinal fluid.

5. The method of claim 1, wherein said method comprises determining whether or not said biological fluid from said mammal contains an elevated level of said TDP-43 polypeptide.

6. The method of claim 5, wherein said elevated level of said TDP-43 polypeptide is greater than 10 ng/mL.

7. The method of claim 1, wherein said method comprises determining whether or not said biological fluid from said mammal contains an elevated level of said TDP-43 polypeptide cleavage product.

8. The method of claim 7, wherein said elevated level of said TDP-43 polypeptide cleavage product is greater than 10 ng/mL.

9. The method of claim 1, wherein said method comprises obtaining said biological fluid from said mammal.

10. The method of claim 1, wherein said mammal comprises said elevated level, and wherein said method comprises classifying said mammal as having said neurodegenerative disease.

11. The method of claim 1, wherein an anti-TDP-43 polypeptide antibody is used to determine whether or not said biological fluid from said mammal contains said elevated level.

12. The method of claim 1, wherein said TDP-43 polypeptide cleavage product is about 25 kD.

13. The method of claim 1, wherein said TDP-43 polypeptide cleavage product is about 35 kD.

14. The method of claim 1, wherein an antibody is used to determine whether or not said biological fluid from said mammal contains said elevated level.

15. The method of claim 14, wherein said antibody recognizes a human TDP-43 polypeptide cleavage product that is about 25 kD.

16. The method of claim 15, wherein said antibody does not recognize a full length human TDP-43 polypeptide.

17. The method of claim 15, wherein said antibody was produced using the sequence set forth in SEQ ID NO:3.

18. An antibody comprising the ability to recognize a human TDP-43 polypeptide cleavage product that is about 25 kD, wherein said antibody does not recognize a full length human TDP-43 polypeptide.

19. The antibody of claim 18, wherein said antibody was produced using the sequence set forth in SEQ ID NO:3.
